(19) United States
(12) Patent Application Publication
(10) Pub. No.: US 2024/0254454 A1
(43) Pub. Date: Aug. 1, 2024

(54) RECOMBINANT AAV VECTORS EXPRESSING OSTEOPROTECTIVE GENES, INCLUDING HAS2 AND LUBRICIN, USEFUL IN THE TREATMENT OF OSTEOARTHRITIS AND RELATED JOINT CONDITIONS IN MAMMALS

(71) Applicant: Genzyme Corporation, Cambridge, MA (US)

(72) Inventors: Monica DIAS FIGUEIREDO, Athens, GA (US); Sirkka RM KYOSTIO-MOORE, Ashland, MA (US); Patricia BERTHELETTE, Uxbridge, MA (US)

(21) Appl. No.: 18/407,298

(22) Filed: Jan. 8, 2024

Related U.S. Application Data
(62) Division of application No. 16/524,645, filed on Jul. 29, 2019, now Pat. No. 11,905,531.
Application No. 16/524,645 is a continuation of application No. 15/405,992, filed on Jan. 13, 2017, now abandoned.
(60) Provisional application No. 62/278,243, filed on Jan. 13, 2016.

(51) Int. Cl. C12N 7/00; A61K 38/45; A61K 48/00; C07K 14/78; C12N 9/10; C12N 15/86

(52) U.S. Cl. CPC C12N 7/00; A61K 38/45; A61K 48/005; C07K 14/78; C12N 9/1051; C12N 15/86; C12Y 204/01212; C12N 2750/14111; C12N 2750/14121; C12N 2750/14143

(57) ABSTRACT

The present disclosure relates to recombinant viral vectors, to pharmaceutical compositions comprising such recombinant vectors, and to methods for prevention and treatment of osteoarthritis in mammals. In particular, this disclosure provides adeno-associated virus (AAV) vectors capable of expressing, in a host, osteoprotective/chondroprotective bioactive proteins, including hyaluronan synthase 2 (HAS2) and lubricin (PRG4). Methods of production of these AAV are provided, as are methods of treatment of osteoarthritis in mammalian joints, by the long-term gene expression of osteoprotective/chondroprotective proteins, including HAS2 and PRG4, in both synovial and chondrocyte cells.

### CROSS-REFERENCE TO RELATED APPLICATIONS

This application is a divisional of U.S. patent application Ser. No. 16/524,645, filed Jul. 29, 2019, which is a continuation of U.S. patent application Ser. No. 15/405,992, filed Jan. 13, 2017 (now abandoned), which claims priority to U.S. provisional application No. 62/278,243, filed Jan. 13, 2016, each of which is herein incorporated by reference in their entirety.

### REFERENCE TO AN ELECTRONIC SEQUENCE LISTING

The contents of the electronic Sequence Listing (159792017310SEQLIST.xml; size 35,824 bytes; and created on Jan. 3, 2024) is herein incorporated by reference in its entirety.

### FIELD OF THE INVENTION

The present invention relates to recombinant vectors, to pharmaceutical compositions comprising such recombinant vectors, and to methods for prevention and/or treatment of acute and/or chronic joint conditions, including osteoarthritis, in mammals. In particular, the invention relates to adeno-associated virus (AAV) vectors capable of expressing, in a host, a bioactive polypeptide belonging to the hyaluronan synthase 2 (HAS2) and lubricin (PRG4) family of proteins. Accordingly, the invention relates to the field of genetic engineering and provides an adeno-associated virus (AAV)-based biological delivery and expression system for use in the treatment of osteoarthritis in human or mammalian joints by long-term gene expression of HAS2 and LUB in synovial and chondrocyte cells.

### SUMMARY OF THE INVENTION

Osteoarthritis (OA) is a degenerative joint disease that occurs in mammalian joints and constitutes a severe economical and medical problem (Matthews, G. L., and Hunter, D. J. (2011). Expert Opin. Emerging Drugs 1-13; Brooks P M. Curr Opin Rheumatol 2002; 14: 573-577). Cartilage is the tough connective tissue that covers the ends of bones in joints. It provides for a relatively frictionless, highly lubricated surface between rigid bones and allows for a smooth movement. During OA development, cartilage is partially or completely lost due to abnormal or excessive wearing, which leads to exposed bone ends that rub against each other resulting in inflammation, pain, swelling or loss of mobility. Currently, the detailed reasons for the initial cartilage loss that leads to OA are not known, but there is a strong correlation between the incidence and age, obesity and joint overuse such as excessive athletic activity.

In dogs, osteoarthritis (OA) is one of the most common causes of lameness, and it is estimated to affect approximately 20 percent of dogs over the age of one year. No curative treatment is currently available for OA, so medical treatment has largely targeted symptom alleviation rather than re-establishment of cartilage. An analgesic treatment usually involves steroids and non-steroidal anti-inflammatory drugs (NSAIDS), which have shown efficacy in the treatment of OA for some decades. However, while these drugs can suppress joint inflammation, many of them are known to have deteriorating effects on the cartilage, which further worsens the underlying process of OA development. In addition to traditional analgesic and anti-inflammatory therapies, direct administration of naturally-occurring osteoprotective compounds has been used to alleviate OA symptoms, varying degrees of success. For example, hyaluronic acid (HA) has been widely used to restore viscoelasticity and lubrication of affected joints. Also, polysulphated glycosaminoglycans (PSGAGs), injected via the intra-articular or intramuscular route, and orally-administered glucosamine and chondroitin sulphate have shown some efficacy.

However, the foregoing drugs must be administered frequently, sometimes even in combination with each other, to achieve meaningful alleviation of symptoms. These frequent joint injections are laborious/costly, bear the risk for infections, and cause a great deal of stress for the patient or animal. And while surgical approaches have also been developed, these have generally shown low efficacy in dogs and horses, and are typically only performed in severe advanced-stage subjects.

In addition to delivering supplements/drugs, several groups have attempted to improve OA symptoms by delivering viscoelastic/viscoprotective polypeptides, nucleic acids encoding same, or polypeptides or nucleic acids capable of expressing in a host the means for producing a viscoprotective protein (e.g., an enzyme). Approaches of greater interest include the use of lubricin polypeptides (Flannery, U.S. Pat. No. 7,642,236 B2), tribonectins (U.S. Pat. No. 7,618,914 B2, to Rhode Island General Hospital), and hyaluronan synthase (U.S. Pat. No. 6,423,514, to Millennium Pharmaceuticals).

Some of these efforts may be characterized as “gene therapy,” the basic concepts of which are well established (Evans C H, Robbins P D. Gene therapy for arthritis, In: Wolff J A (ed.). Gene Therapeutics: Methods and Applications of Direct Gene Transfer. Birkhauser: Boston, 1994, pp 320-343). Recently, one group has tried to treat osteoarthritis by the in vivo delivery of the interleukin-1 receptor antagonist (Il-1Ra) gene (US 2015/0031083 A1, to Baylor College of Medicine; and see Frisbie, D D et al., Gene Therapy (2002)).

The Arthrogen company has used AAV5 to express human interferon beta (to reduce inflammatory cytokine) in the context of rheumatoid arthritis (RA). Unlike OA, inflammatory signaling plays a significant role in the pathology of RA, and so blocking this signal is a key therapeutic approach. That said, another group, pursuing a possible link between inflammation and OA, used recombinant AAV2 to express an IL1 receptor antagonist, in the context of equine OA (Goodrich et al., Molecular Therapy-Nucleic Acids (2013) 2, e70).

However, none of these approaches has proven universally effective, and there remain significant unmet needs as to relief of pain and suffering for OA patients. It follows that there is a clear and yet unmet medical need for more efficacious and sustained treatments that are at the same time also cost effective in the long run.

Accordingly, as described in detail herein, Applicants successfully demonstrated for the first time that recombinant adeno-associated virus (rAAV) vectors could deliver cDNAs encoding therapeutic agents by a single intra-articular injection into a mammalian joint to facilitate local and continuous production of the agent in vivo in synoviocytes and chondrocytes.

Applicants have also isolated and sequenced, for the first time, a full length canine lubricin CDNA (SEQ ID NO:4).

The present invention provides rAAV vectors that express in vivo, in a mammalian host, therapeutically effective amounts of osteo-protective and/or osteo-regenerative gene products.

In aspects, the rAAV may contain cDNA encoding for an agent with disease-modifying, lubricating, anti-inflammatory and pain relief properties.

In aspects, the rAAV vector is a vector derived from an AAV serotype, including without limitation, AAV1, AAV2, AAV3, AAV4, AAV5, AA6, AAV7, AAV8, AAV9, AAVrh.8, and AAVrh.10. In some embodiments, the nucleic acid in the AAV comprises an ITR of AAV1, AAV2, AAV3, AAV4, AAV5, AAV6, AAV7, AAV8, AAV9, AAVrh.8, or AAVrh.10. In further embodiments, the rAAV particle comprises capsid proteins of AAV1, AAV2, AAV3, AAV4, AAV5, AA6, AAV7, AAV8, AAV9, AAVrh.8, or AAVrh.10. In some embodiments, the ITR and capsid are derived from the same AAV serotype. In other embodiments, the ITR and capsid are derived from different AAV serotypes. In some embodiments, the rAAV vectors may be AAV2 or AAV5 capsid serotypes. In some related embodiments, the rAAV2 and rAAV5 vectors contain at least one ITR that is derived from AAV2.

In aspects embodiments, the rAAV vectors encode hyaluronic acid synthase-2 (HAS2) or variant thereof. In some embodiments, the HAS2 is human HAS2. In other embodiments, the HAS2 is canine HAS2. In some embodiments, the HAS2 is codon-optimized HAS2.

Glycosaminoglycan hyaluronan acid (HA) is a non-sulfated glycosaminoglycan consisting of repeating glucuronic acid and N-acetylglucosamine residues linked by beta-1-3 and beta-1-4 glycosidic bonds. It provides multiple biological functions including wound healing, cell migration, malignant transformation and tissue turnover. HA is synthesized by various cell types including endothelial cells, fibroblasts and smooth muscle cells and has been detected in tissues such as connective, epithelial and neural tissues. In the joint space, HA is made by synoviocytes that secrete HA into synovial fluid as well as by chondrocytes. The synovial fluid HA provides lubrication, tissue hydration, structural integrity and scaffold for matrix proteins and biomechanics as well as plays a role in joint homeostasis. The biological effects of HA in the joint are determined by its concentration and molecular weight. It can be synthesized ranging from 5000 Da to 10,000 000 Da. The lower molecular weight HA (<500 kDa) is involved in receptor-mediated activation of angiogenesis, malignancy and inflammation while the higher molecular weight HA provides lubrication in the joint.

The sizes and concentration of joint HA is regulated both by its rate of synthesis and degradation. Degradation of HA is mediated by hyaluronidases that cleave HA into smaller fragments that drain into lymphatic system for clearance. Three enzymes, named as hyaluronan synthase (HAS) 1, 2, and 3 have been described for HA production and reside at the inner surface of plasma membrane of synoviocytes. Of these, HAS2 has been shown to be responsible for production of high molecular weight HA (Itano et al. 1999). It has been reported that the high molecular weight HA levels are decreased in osteoarthritic joints both in human patients and animal OA models (Plickert et al., 2013). This is likely due to both reduced synthesis of HA and increased degradation of HA by the hyaluronidases. Of the HA synthases, HAS2 and 3 are expressed in human cartilage and of these, HAS2 expression is reduced in human OA. HA levels are also reduced due to increased levels of HA degrading enzyme, hyaluronidase 2 (Yoshida et al.). The HAS2 promoter has been reported to be responsive to various pro- and anti-inflammatory mediators with conflicting effects reported. These include TGFβ, primary epidermal growth factor, TNF alpha and retinoic acid (Guo, Kanter el al, 2007, Hyc et al., 2009). Down-regulation by inflammatory mediators in diseased joint is expected to reduce HAS2 expression resulting decreased HA levels and can differentially affect various HAS isoforms (David-Raoudi et al., 2009). In contrast, mechanical stimulation (Momberger et al. 2005) or cartilage components such as chondroitin sulfate have been reported to stimulate HA production (Momberger et al., 2005, David-Raoudi et al., 2009). HA production results in pericellular location as well as secretion into extracellular space. It is not clear what regulates the extent of secretion. However, typically about 80% of the HA is secreted while the remainder remains associated with producing cells. This cell-associated HA is important for assembly of matrix proteins; blocking HAS2 synthesis results in decreased cell-associated matrix and increased release of proteoglycans (i.e. aggrecan) into media furthermore confirming major role of HAS2 as major enzyme synthesizing HA in chondrocytes (Nishida et al. 1999).

In aspects embodiments, the rAAV vectors encode lubricin or a variant thereof. In some embodiments, the lubricin is human lubricin. In other embodiments, the lubricin is canine lubricin. In some embodiments, the lubricin is codon-optimized lubricin.

Lubricin (PRG4), which is a large mucin glycoprotein made by joint synovial lining cells and cartilage chondrocytes and provides a protective lubrication for cartilage surfaces (Flannery 1999, Schmidt 2001, Waller 2013). Lubricin along with HA is also an important lubricant in the synovial fluid providing shock-absorbing properties. Lack of lubricin in mouse models and in a rare human genetic disease results in cartilage degeneration characteristic of osteoarthritis (OA) (Rhee 2005, Ruan 2013). Decreased synthesis of lubricin has also been demonstrated in human OA patients and various animal OA models (Elsaid, 2008). Intra-articular lubricin supplementation with recombinant lubricin has been shown to improve cartilage pathology (Flannery 2009).

In some embodiments, the rAAV vectors may be AAV2 or AAV5 capsid serotypes encoding canine codon-optimized hyaluronic acid synthase-2 (HAS2).

In aspects, the rAAV vector is administered via intra-articular delivery. In embodiments, the rAAV are administered via a single intra-articular delivery. In another embodiment, following the intra-articular administration, in vivo production and secretion of the cognate therapeutic agent from the rAAV-transduced cells may persist for at least about 6 months.

In some embodiments, the rAAV vector contains an expression cassette containing an ubiquitous promoter and a codon-optimized and species-matched transgene (see e.g. FIG. 1A).

In another aspect, the disclosure provides a method of using the rAAV vectors to express in vivo in an animal's joint osteo-protective and/or osteo-regenerative gene products.

All references cited herein, including patent applications and publications, are incorporated by reference in their entirety.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1A is a diagram showing the HAS expression cassette in a plasmid (top) and rAAV viral vector (bottom).

FIG. 1B is a graph showing production of HA from cells transfected with cHAS2 expression plasmid. The cHAS expression plasmid was transfected into 293 cells and conditioned media harvested 3 days later and HA levels in the media were quantitated using an HA-binding protein-based detection system. Abbreviations: CBAcHAS2, HAS2 expression plasmid. EGFP, EGFP expression plasmid. untransfected, negative control cells. “Optimem”=cells grown in serum-free media. “Complete”=cells grown in serum-containing media.

FIG. 1C are images of agarose gels used to separate components of conditioned media from the cHAS-transfected 293 cells (+ or − hyaluronidase treatment for 24 h), confirming expression of HA. Abbreviations: CM, conditioned media. MDa, molecular weight size or markers.

FIG. 2A is a graph showing rAAV vector production from a small-scale packaging of cHAS2 into rAAV2 and AAV5 vectors using triple transfection method. Packaging of EGFP expression cassette into AAV2 is shown as positive control and packing of cHAS in the absence of capsid plasmid as a negative control. The rAAV yield is shown as the amount of DNA resistant particles (DRP) per cell.

FIG. 2B is a graph showing rAAV vector yields from a large scale vector production by triple transfection. Examples of total titers obtained for multiple vector lots obtained are shown.

FIG. 2C is a graph showing the potency of AAV2/HAS2 vectors in vitro. The 293 cells were infected by various MOIs and HA levels in conditioned media were quantitated 3 days later.

FIG. 2D is a graphs showing the potency of AAV5/HAS2 vectors in vitro.

FIG. 3A is a graph showing changes in bodyweight post intra-articular injection of rAAV/HAS2 vectors in normal canine joints.

FIG. 3B is a graph showing Cartilage scoring.

FIG. 3C is a graph showing synovial scoring.

FIG. 4A is a diagram showing the locations of sample collection for the canine synovium rAAV vector quantitation.

FIG. 4B is a graph showing quantitation of vector genome copies in synovial sample #3.

FIG. 4C is a graph showing vector genome copies in synovial sample #1. The vector dose for AAV2 and AAV5 is shown as L=low, M=medium and H=high (as in FIGS. 3A-C). All tissue samples were collected 28 days after rAAV vector delivery and analyzed by qPCR to BGHpA.

FIG. 5A is a graph showing vector derived cHAS expression in synovium sample #3.

FIG. 5B is a graph showing vector mRNA in synovial sample #1.

FIG. 5C is a graph showing vector genome and vector derived mRNA in each individual dog analyzed using synovial sample #3. Vector dose for AAV2 and AAV5 is shown as L=low, M=medium and H=high. All tissue samples were collected 28 days after rAAV vector delivery and analyzed by qPCR to BGHpA.

FIG. 6A is a diagram showing the locations of femoral condyle and tibial plateau samples collected to detect rAAV vector and mRNA in cartilage.

FIG. 6B is a graph showing vector genome and vector derived mRNA in each individual dogs analyzed using femoral condyle sample #1. Additionally, vector genome copies in contralateral (un-injected right joint) are shown (with the exception of sample #22, which was not tested).

FIG. 6C is a graph showing the average of vector genome (injected and un-injected joints) and mRNA copies in each group. Vector dose for AAV2 and AAV5 is shown as L=low, M=medium and H=high (see FIG. 3). All tissue samples were collected 28 days after rAAV vector delivery and analyzed by qPCR to BGHpA.

FIG. 6D is a graph showing the average of vector genome (PBS or vector-injected joints) and mRNA copies in tibial plateau cartilage in each group.

FIG. 7A is a graph showing vector genomes in synovium (samples #3 and #1) and cartilage (femoral condyle and tibial plateau) in each treatment group in tissues collected from the left stifle joints. The values shown in FIGS. 7A and 7B represent group average±standard deviation (n=5/group).

FIG. 7B is a graph showing quantitation of vector genomes and mRNA from the rAAV5/HAS2 vector in various tissues.

FIG. 8A is a graph showing the HA levels in canine synovial fluids. The HA levels were quantitated in SF samples collected on days −7 (baseline) and day 28. The HA levels in each animal were normalized to baseline levels and expressed as % of HA on day 28 compared to week before vector administration.

FIG. 8B is a graph showing the HA levels in canine synovial fluids at day −7 (baseline) and day 28. Arrows indicate animals with higher HA levels on day 28 compared to baseline (before treatment).

FIG. 9 shows the complete amino acid sequence of canine lubricin (SEQ ID NO: 5). The boxed area indicates location of exon 6 (mucin domain). The underlined amino acids (378 to 782) are deleted in the shortened canine lubricin (hereinafter “cLub1” or “cLubico”), and the locations of KEPAPTT-like repeats (potential O-linked glycosylation sites) are in bold. When a sequence name ends in “co,” it means the cDNA sequence has been “codon-optimized.” Similarly, “nonco” means non-codon-optimized.

FIG. 10 is a diagram showing the plasmids generated and used in the experiments. The plasmids contain a shortened (i.e. an engineered internal deletion), codon-optimized canine lubricin sequence (cLubico), promoter (minCBA or CBA) and a BGHpA site. Some constructs contain a N- or C-terminal His-tag (C-term) and a modification of ATG (potential start codons) removed from the intron sequence. A pre-viral AAV lubricin plasmid also contains flanking ITR sequences at both ends.

FIG. 11A is a graph showing mRNA copies/cell produced when the minCBA cLub1, CBA CLUB1 and CBH cLub-nonco constructs were transfected into 293 cells.

FIG. 11B is a graph showing mRNA copies/cell produced when the ΔATG/6His/N′, 6His/N′, 6His/C′, WT cLub, and EGFP constructs were transfected into 293 cells.

FIG. 12A is an anti-lubricin Western blot showing levels of secreted lubricin in concentrated media (plasmids described above). Canine synovial fluid was used as a positive control.

FIG. 12B is a Western blot showing lubricin production from pre-viral lubricin expression plasmids. Two clones were analyzed and compared to expression obtained with the minCBA-cLubco plasmid. Untransfected culture media and EGFP-expression plasmid transfected cells were run as negative controls.

FIG. 13A is a graph showing vector yields in small-scale vector production for AAV2 vectors encoding canine lubricin. Two cLub clones (−/+6×His-tag) were analyzed and compared to packaging of EGFP and HAS2 expression cassettes present pre-viral (ITR-containing) plasmids. Negative controls included un-transfected cells and transfections lacking AAV2 capsid expressing plasmid.

FIG. 13B is a graph showing vector yields in small scale vector production for AAV5 vectors encoding canine lubricin. Pre-viral plasmids for EGFP and cLub expression cassettes were transfected together with AAV5 capsid expressing plasmid.

FIG. 14 is an anti-lubricin Western blot showing canine lubricin expression from rAAV5 vector in vitro. Human 293 cells were infected with rAAV5/minCBA-cLub1 at various amounts for 72 h followed by concentration of conditioned culture media. Culture media from AAV5/CBA-EGFP infected cells were used as negative control. Culture media from pre-viral lubricin expression plasmid transfected cells were used as positive control

FIG. 15 is a table presenting of summary of SEQ ID NOs.

FIG. 16 is an alignment of canine and human lubricin.

FIG. 17 is a graph showing HA levels in various time-points in the canine synovial fluid using the MMR model. Synovial fluid was collected a week before OA induction (pre), two weeks after induction and prior to test article administration (day 0) and 57, 112 and 182 days after test article delivery

FIG. 18A is a graph showing rAAV5 vector detection and expression in synovial samples from canine OA joints 182 days after vector administration.

FIG. 18B is a graph showing rAAV5 vector detection and expression in cartilage (femoral condyles) of canine OA joints 182 days after vector administration.

FIG. 18C summarizes rAAV5 vector genome and cHAS2 mRNA detection in synovial and cartilage samples on day 182 in the canine MMR OA model.

FIG. 19 shows safranin-O stained sections of cartilage surfaces obtained from the medial side from one PBS- and two rAAV5/cHAS2-treated canine joints as examples.

### DETAILED DESCRIPTION OF THE INVENTION

Osteoarthritis (OA) is one of the most common causes of lameness in mammals, and in dogs and is estimated to affect approximately 20% of dogs >1 year old. OA is a progressive and degenerative disease resulting in pain, inflammation and reduced joint mobility. Novel safe and efficacious therapies that improve joint lubrication and reduce inflammation and pain are needed to manage OA. As disclosed herein, Applicants discovered that recombinant adeno-associated virus (rAAV) vectors can be used to deliver genes encoding therapeutic agents by a single intra-articular injection with the goal to provide local and continuous production of the agent in the joint. rAAV vectors were generated with AAV2 and AAV5 capsid serotypes and encoding canine codon-optimized hyaluronic acid (HA) synthase-2 (HAS2).

Twenty-two adult healthy dogs, seronegative for AAV2 and AAV5 capsids received rAAV2 (1, 5 and 10×10¹¹ vg/joint), rAAV5 (5×10¹¹ vg/joint) or PBS (control) via intra-articular injection. No adverse clinical signs were observed following the 28-day study. Histopathological analysis showed minimal synovial inflammation in joints treated with rAAV5 and no significant changes in the rAAV2 treatment groups. Vector genomes (VG) were detected in the synovium of all the rAAV-treated joints and in the majority of cartilage samples. The rAAV5 vectors resulted in higher VG detection and mRNA expression compared to rAAV2 in both tissues. A preliminary analysis also showed a trend of increased HA levels in the synovial fluid of the treated joints. In summary, our study demonstrated gene transfer to canine joint tissues and an acceptable safety profile with rAAV2 and rAAV5 vectors encoding HAS2 when administered by single intra-articular injection in a limited number of dogs.

### Canine HA Synthase 2.

In an aspect of the invention, the disclosure provides a recombinant adeno-associated virus (rAAV) vector comprised of AAV capsid and a single-stranded DNA genome. Viral capsids according to the disclosure may confer uptake of the vector into joint cells, with subsequent transport to the cell's nucleus, resulting in expression of a therapeutic gene. In some embodiments, the DNA genome contains one or more AAV inverted terminal repeats (ITRs) flanking one or more expression cassette(s), for expressing in vivo in an animal host the therapeutic gene. In some embodiments of the invention, no viral genes will be present or expressed from the rAAV genome.

In some embodiments of the invention, once the rAAV has been administered to an animal and is taken up by the animal's cells, the rAAV genome will persist as an extrachromosomal episome. In some embodiments, the rAAV of the disclosure may persist long-term in the joint cells; for example, but not limited to more than about 1 month, 2 months, 3 months, 4 months, 5 months, 6 months, 7 months, 8 months, 9 months, 10 months, 11 months, 1 year, 2 years, or 3 years. The episomal rAAV will continue to express, resulting in the production and secretion of the therapeutic agent into synovial fluid, thereby providing a local and continuous production of the agent directly into the joint. The selected transgene product may promote joint health by increasing joint lubrication, reducing pain and cartilage degradation and the like.

In a another aspect of the invention, the disclosure provides a method of treating an animal in need thereof comprising the step of administering to the animal a therapeutically effective amount of a rAAV according to the disclosure. In some embodiments, the method comprises administering to canines the proposed product directly into its affected joint. In some embodiments, a single treatment is sufficient to affect significant improvement in the animal's condition.

In other embodiments, the treatment is repeated. In some embodiments, the treatment is repeated within 2-3 weeks of the first administration, and in other embodiments, the second administration is given greater than 3 weeks after the first. In some related embodiments, the second dose comprises administration of a rAAV with the same therapeutic gene and the rAAV comprises the same serotype capsid as the first treatment, and in other related embodiments, the second dose comprises administration of a rAAV with the same therapeutic gene but the rAAV comprises a different serotype capsid as the first dose. In some embodiments, the rAAV has a serotype 5 capsid. In related embodiments, where repeated administration is desired, the first dose may comprise administration of a rAAV having a serotype 5 capsid and the second dose may comprise administration of a rAAV having a serotype 5 capsid.

In aspects, overexpression of the HAS2 protein in the osteoarthritic joint elevates levels of HA in synovial fluid and improves joint health by increasing lubricating, anti-inflammatory and pain relief properties of HA. Overexpression of HAS2 has been shown to result in elevated levels of HA in the culture media by various cell types in vitro. This has been demonstrated using CHO, 293, and COS cells either as stable transfectant or transient transfection. To provide overexpression of HA in the joint in vivo, the HAS2 cDNA can be delivered to cartilage and/or synovium, the normal sites of HA synthesis by using rAAV vector encoding HAS2 expression cassette. Not wishing to be bound by any theory, gene transfer to cells will provide HAS2 expression cassette for sustained expression of HAS2 and subsequent production of HA. As the therapeutic vector will contain a ubiquitous promoter, it will not be subject to down-regulation by inflammatory mediators present in the osteoarthritic joint unlike the endogenous HAS2 promoter. When the vector is administered by intra-articular injection it can result in transduction of various cell types, the major cell types being synoviocytes. Lastly, the HAS2 protein alone has been shown to be sufficient to synthesize HA and no other associated proteins or components are thought to be necessary for HA production in vitro (Yoshida et al.).

### Canine Lubricin.

In aspects, lubricin production in osteoarthritic joints is increased by intra-articular delivery of recombinant adeno-associated virus (rAAV) vector encoding lubricin as a potential treatment for canine osteoarthritis (OA). Lubricin is a large secreted glycoprotein that functions as a lubricant and protects cartilaginous surfaces in a joint. A Described herein is the discovery and generation of cDNA for a full-length canine lubricin that was used to design a shortened and codon-optimized version of canine lubricin (cLubico). The latter was then used to construct various lubricin expressing plasmids. The plasmids were characterized for lubricin mRNA and protein production after transfection into HEK293 cells. The data showed both production of lubricin mRNA and secreted lubricin from each construct. rAAV vectors were generated with cLubico expression cassette and demonstrated the feasibility of rAAV/cLub1 vector production. HEK293 cells infected with this construct synthesized and secreted canine lubricin.

The methods and compositions described herein can also be used for therapeutic treatment of osteoarthritis. The terms “therapy” or “therapeutic treatment”, as they relate to osteoarthritis, and as they are used herein and in the field of veterinary medicine, relate to treating, or supporting and/or accelerating treatment of, subjects that are already suffering from, or are recovering from (e.g., are in the recovery phase) osteoarthritis, or treatments aimed at slowing down and/or reversing cartilage loss in subjects diagnosed as having, or at being at risk of, osteoarthritis. A critical objective of therapy is to reduce the risk of an evolution towards cartilage and bone loss. As used herein, a subject is said to suffer from osteoarthritis, or be at risk of developing osteoarthritis, if the subject is reasonably expected to suffer a progressive cartilage loss associated with osteoarthritis. Whether a particular subject suffers of osteoarthritis, or is at risk of developing osteoarthritis, can readily be determined by one with ordinary skill in the relevant veterinary or medical art.

The methods and compositions described herein may also be used for preventative treatment of osteoarthritis. The terms “prevention”, “prophylaxis”, “preventative treatment” and “prophylactic treatment”, as they relate to osteoarthritis, and as they are used herein and in the field of human and veterinary medicine, relate to the treatment of either healthy subjects or subjects suffering from an unrelated disease, but who are considered to be at risk of osteoarthritis.

Described herein are therapies and preventative treatments for osteoarthritis that utilize pharmaceutical compositions comprising vectors capable of expressing HAS or Lubricin polypeptides in vivo and methods and compositions for inducing a sustained increase in joint hyaluronic acid or lubricin concentrations, to reduce or eliminate cartilage loss.

As used herein, a pharmaceutical composition is said to have “therapeutic efficacy”, or to be “therapeutically effective”, if administration of that amount of the composition is sufficient to cause a significant improvement of the clinical signs or measurable markers of the disease in a mammalian subject suffering from osteoarthritis. As used herein, a pharmaceutical composition is said to have “prophylactic efficacy” or to be “an effective”, if administration of that amount of the composition is sufficient to prevent the development of osteoarthritis in a subject.

Also described herein is a vector capable of expressing, in vivo in a host, a HAS or lubricin polypeptide, or variants or fragments or combinations thereof. In embodiments, the HAS or lubricin polypeptides for use in the present invention are genetically matched to the intended target species (e.g., vectors encoding canine HAS2 are delivered to canines suffering from OA).

By way of illustration of “variants,” “derivatives,” and the like described herein include, but are not limited to, HAS and lubricin variants, derivatives, and the like that are encoded by nucleotide sequences that are not exactly the same as the nucleotide sequences disclosed herein, but wherein the changes in the nucleotide sequences do not change the encoded amino acid sequence, or result in conservative substitutions of amino acid residues, deletion of addition of one or a few amino acids, substitution of amino acid residues by amino acid analogues that do not significantly affect the properties of the encoded polypeptides (e.g., the variant or derivative has more than about 10%, 20%, 30%, 40%, 50%, 60%, 70%, 80%, 90%, 95%, 99% or 100% of the desired activity of wild type polypeptide), and the like. Examples of conservative amino acid substitutions include glycine/alanine substitutions; valine/isoleucine/leucine substitutions; asparagine/glutamine substitutions; aspartic acid/glutamic acid substitutions; serine/threonine/methionine substitutions; lysine/arginine substitutions; and phenylalanine/tyrosine/tryptophan substitutions. Other types of substitutions, variations, additions, deletions and derivatives that result in functional HAS or lubricin derivatives, are also described herein, and one of skill in the art would readily know how to make, identify, or select such variants or derivatives, and how to test for HAS or lubricin activity of those variants or derivatives. One of skill in the art may optimize the expression of the HAS or lubricin polypeptides of the invention; for example, but not limited to removing cryptic splice sites, adapting the codon usage by introducing a Kozak consensus sequence before the start codon, changing the codon usage or combination thereof to improve expression.

The vector for use in the present invention may comprise a nucleic acid sequence encoding a canine HAS2 polypeptide comprising the amino acid sequence set forth in SEQ ID NO: 2. In some embodiments, the canine HAS2 polypeptide is a canine HAS2 variant having at least 90%, at least 91%, at least 92%, at least 93%, at least 94%, at least 95%, at least 96%, at least 97%, at least 98% or at least 99% homology or identity to SEQ ID NO: 2.

The vector for use in the present invention may comprise a nucleic acid sequence encoding a canine lubricin polypeptide comprising the amino acid sequence set forth in SEQ ID NO: 7. In some embodiments, the canine lubricin polypeptide is a canine lubricin variant having at least 90%, at least 91%, at least 92%, at least 93%, at least 94%, at least 95%, at least 96%, at least 97%, at least 98% or at least 99% homology or identity to SEQ ID NO: 7.

Sequence identity or homology may be determined by comparing the sequences when aligned so as to maximize overlap and identity while minimizing sequence gaps. In particular, sequence identity may be determined using any of a number of mathematical algorithms. A non-limiting example of a mathematical algorithm used for comparison of two sequences is the algorithm of Karlin & Altschul, Proc. Natl. Acad. Sci. USA 1990, 87, 2264-2268, modified as in Karlin & Altschul, Proc. Natl. Acad. Sci. USA 1993, 90, 5873-5877.

Another example of a mathematical algorithm used for comparison of sequences is the algorithm of Myers & Miller, CABIOS 1988, 4, 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988, 85, 2444-2448.

In general, comparison of amino acid sequences may be accomplished by aligning an amino acid sequence of a polypeptide of a known structure with the amino acid sequence of a the polypeptide of unknown structure. Amino acids in the sequences are then compared and groups of amino acids that are homologous are grouped together. This method detects conserved regions of the polypeptides and accounts for amino acid insertions and deletions. Homology between amino acid sequences can be determined by using commercially available algorithms (see also the description of homology above). In addition to those otherwise mentioned herein, mention is made too of the programs BLAST, gapped BLAST, BLASTN, BLASTP, and PSI-BLAST, provided by the National Center for Biotechnology Information. These programs are widely used in the art for this purpose and can align homologous regions of two amino acid sequences.

In all search programs in the suite, the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

The terms “protein”, “polypeptide” and “polypeptide fragment” are used interchangeably herein to refer to polymers of amino acid residues of any length.

As used herein the term “polynucleotide” is used to refer to a polymeric form of nucleotides of any length, which contain deoxyribonucleotides or ribonucleotides.

The term “vector”, as used herein, refers to a recombinant DNA or RNA plasmid or virus that comprises a heterologous polynucleotide to be delivered to a target cell, such as in vivo. The heterologous polynucleotide may comprise a sequence of interest for purposes of therapy, and may optionally be in the form of an expression cassette. As used herein, a “vector” need not be capable of replication in the ultimate target cell or subject.

The term “recombinant” as used herein means a polynucleotide semisynthetic, or synthetic origin, which either does not occur in nature or is linked to another polynucleotide in an arrangement not found in nature.

The term “heterologous” as used herein derived from a genetically distinct entity from the rest of the entity to which it is being compared. For example, a polynucleotide may be placed by genetic engineering techniques into a plasmid or vector derived from a different source, and is thus a heterologous polynucleotide. A promoter removed from its native coding sequence and operably linked to a coding sequence other than the native sequence is accordingly a heterologous promoter.

The polynucleotides for use according to the invention may comprise additional sequences, such as additional coding sequences within the same transcription unit, controlling elements such as promoters, ribosome binding sites, transcription terminators, polyadenylation sites, additional transcription units under control of the same or different promoters, sequences that permit cloning, expression, homologous recombination, and transformation of a host cell, and any such construct as may be desirable to provide embodiments of this invention.

In an aspect, the disclosure provides a method of treating a mammalian subject suffering from, or at risk of developing, osteoarthritis (OA), comprising administering to said mammalian subject a therapeutically effective amount of an adeno-associated virus (AAV) containing a nucleic acid sequence encoding an osteo-protective or osteo-regenerative polypeptide and operably linked to a promoter, wherein the polypeptide is expressed in vivo in the mammalian subject and, in an amount effective to alleviate or prevent the symptoms of OA. In some embodiments, the administration is via the intra-articular route.

In some embodiments, the polypeptide may encode a hyaluronic acid synthase (HAS), including HAS2 (HAS2), a lubricin, an Interleukin-1 Receptor (IL-1R) antagonist, an Insulin-like growth factor 1 (IGF-1), a fibroblast growth factor 2 (FGF-2), a Transforming growth factor beta 1 (TGFB1), a Bone Morphogenetic protein 7 (BMP7), a Glucosamine-fructose-6-phosphate aminotransferase (GFAT), an Interleukin 10 (IL-10), a heme oxygenase-1 HO-1, biologically active truncations thereof, or combinations thereof. In an embodiment, the mammalian subject may be a human, a canine or a feline. In a particular embodiment, the subject is a canine.

In some embodiments, the mammalian subjects are suffering from, or are at risk of developing chronic osteoarthritis.

In other embodiments, the polypeptide is canine HAS2 or canine lubricin. In a some embodiments, the nucleic acid sequence encoding the HAS2 polypeptide has a sequence having at least 90% identity to the sequence as set forth in SEQ ID NO: 3, or the nucleic acid sequence encoding the lubricin polypeptide has a sequence having at least 90% identity to the sequence as set forth in SEQ ID NO: 6.

In some embodiments, the HAS2 polypeptide comprises the amino acid sequence set forth in SEQ ID NO: 2. In some embodiments, the HAS2 polypeptide has an amino acid sequence selected from a polypeptide having at least 90% identity to the sequence as set forth in SEQ ID NO: 2, a fragment, a variant, and a homolog thereof having, each exhibiting HAS activity in vivo in the subject. “HAS activity” means production of biologically active hyaluronic acid.

In some embodiments, the AAV vector comprises from 5′ to 3′ the following elements: 5′ AAV ITR, stuffer, CBA, intron (IN), cHAS2 codon-optimized cDNA, polyadenylation signal (pA), and 3′ AAV ITR.

In some embodiments, the lubricin polypeptide comprises the amino acid sequence set forth in SEQ ID NO: 7. In other embodiments, the lubricin polypeptide has an amino acid sequence selected from a polypeptide having at least 90% identity to the sequence as set forth in SEQ ID NO: 7, a fragment, a variant, and a homolog thereof having, each exhibiting lubricin activity in vivo in the subject. “Lubricin activity” means providing lubrication in substantially the same way, and substantially to the same extent, as endogenously-produced lubricin. Such lubricating activity may be measured according to techniques known in the art (see e.g. Swan, D A et al. Biochem J. 1985 Jan. 1; 225(1): 195-201).

In some embodiments, the promoter may be selected from the group consisting of a CMV IE promoter, a RSV promoter, an HSV-1 TK promoter, a SV40 early promoter, a SV40 late promoter, an adenovirus major late promoter, a phosphoglycerate kinase gene promoter, a metallothionein gene promoter, an α-1 antitrypsin gene promoter, an albumin gene promoter, a collagenase gene promoter, an elastase I gene promoter, a CBA promoter, a β-actin gene promoter, a β-globin gene promoter, a γ-globin gene promoter, an α-fetoprotein gene promoter, and a muscle creatine kinase (CK) gene promoter.

In still other embodiments, the AAV comprises an AAV2 or an AAV5 capsid.

In another aspect, the disclosure provides a method of increasing the production of hyaluronic acid in both the chondrocytes and/or synoviocytes of a mammal (e.g., human or canine animal). In an embodiment, the method may comprise the steps of administering the recombinant AAV (“rAAV”) comprising a rAAV vector genome, wherein the rAAV vector genome comprises nucleic acid encoding a HAS2 to a mammal (e.g., human or canine), allowing sufficient time for the HAS2 enzyme to be expressed and subsequently catalyze the production of additional hyaluronic acid, thereby increasing the level of hyaluronic acid in the mammal.

The disclosure also provides a method of increasing the production of a lubricin polypeptide in both the chondrocytes and/or synoviocytes of a mammal (e.g., human or canine animal). In an embodiment, the method may comprise the steps of administering the rAAV comprising a rAAV vector genome, wherein the rAAV vector genome comprises nucleic acid encoding a lubricin to a mammal (e.g., human or canine animal), allowing sufficient time for the lubricin to be expressed, thereby increasing the level of lubricin in the canine.

In an embodiment, the HAS2 is produced in sufficient quantity following administration of an rAAV comprising nucleic acid encoding a HAS2 to treat or prevent the symptoms of OA in a mammal (e.g., a human or a canine).

In another embodiment, the lubricin is produced in sufficient quantity following administration of an rAAV comprising nucleic acid encoding lubricin to treat or prevent the symptoms of OA in a mammal (e.g., a human or a canine).

In an embodiment, the HA levels are restored to levels found in healthy a mammal (e.g., a human or a canine). The skilled person may consult a variety of references to understand what levels of HA are found in healthy animals (e.g., Smith, G N et al. Arthritis Rheum. 1998; 41:976-985; Balazs E et al. Disorders of the Knee. Philadelphia: J B Lippincott; 1982. pp. 61-74).

In another embodiment, the lubricin levels are restored to levels found in healthy a mammal (e.g., a human or a canine).

In another aspect, the disclosure provides a method of treating a canine suffering from, or at risk of developing, OA, comprising, administering to said canine a therapeutically effective amount of an AAV vector containing a nucleic acid sequence encoding an HAS2 or lubricin polypeptide operably linked to a promoter. In another embodiment, the disclosure provides a method of treating a human suffering from, or at risk of developing, OA, comprising, administering to said human a therapeutically effective amount of an AAV vector containing a nucleic acid sequence encoding a HAS2 or lubricin polypeptide operably linked to a promoter.

In some embodiments, the nucleic acid sequence encoding the HAS2 polypeptide has at least 90% identity to the nucleic acid sequence as set forth in SEQ ID NO: 3, or the nucleic acid sequence encoding the lubricin polypeptide has at least 90% identity to the nucleic acid sequence as set forth in SEQ ID NO: 6.

In some embodiments, the AAV encodes a HAS2 polypeptide comprising an amino acid sequence set forth in SEQ ID NO: 2 or comprises an amino acid sequence at least 90% identical to the amino acid sequence set forth in SEQ ID NO: 2. In some embodiments, the AAV encodes a lubricin polypeptide comprising an amino acid sequence set forth in SEQ ID NO: 7 or comprises an amino acid sequence at least 90% identical to the amino acid sequence set forth in SEQ ID NO: 7.

In any embodiment, the promoter may be selected from a CMV IE promoter, a RSV promoter, an HSV-1 TK promoter, a SV40 early promoter, a SV40 late promoter, an adenovirus major late promoter, a phosphoglycerate kinase gene promoter, a metallothionein gene promoter, an α-1 antitrypsin gene promoter, an albumin gene promoter, a collagenase gene promoter, an elastase I gene promoter, a β-actin gene promoter, a CBA promoter, a β-globin gene promoter, a γ-globin gene promoter, an α-fetoprotein gene promoter, and a muscle creatine kinase gene promoter.

In another aspect, the disclosure provides a method of preventing the development of OA in a mammalian subject at risk thereof, comprising, administering to said canine a prophylactically effective amount of a rAAV comprising a rAAV vector genome comprising a nucleic acid sequence encoding a HAS2 or lubricin polypeptide operably linked to a promoter. In an embodiment, the nucleic acid sequence encoding the HAS2 polypeptide has at least 90% identity to the nucleic acid sequence as set forth in SEQ ID NO: 3, or the nucleic acid sequence encoding the lubricin polypeptide has at least 90% identity to the nucleotide sequence set forth in SEQ ID NO: 6. In another embodiment, the nucleic acid encodes a HAS2 polypeptide comprising the amino acid sequence set forth in SEQ ID NO: 2, or the nucleic acid encodes a lubricin polypeptide comprising the amino acid sequence set forth in SEQ ID NO: 7. In an embodiment, the promoter may be selected from the group consisting of a CMV IE promoter, a RSV promoter, an HSV-1 TK promoter, a SV40 early promoter, a SV40 late promoter, an adenovirus major late promoter, a phosphoglycerate kinase gene promoter, a metallothionein gene promoter, an α-1 antitrypsin gene promoter, an albumin gene promoter, a collagenase gene promoter, an elastase I gene promoter, a β-actin gene promoter, a β-globin gene promoter, a γ-globin gene promoter, an α-fetoprotein gene promoter, and a muscle creatine kinase gene promoter. In some embodiments, the rAAV vector comprises CBA-cHAS2co-BGH. In other embodiments, the rAAV vector comprises pITR/minCBA-HIb-cLubico-BGH.

In another aspect, the disclosure provides a recombinant plasmid vector comprising a nucleic acid sequence encoding a canine HAS2 or lubricin polypeptide operably linked to a promoter. In some embodiments, the nucleic acid sequence encoding the HAS2 polypeptide has at least 90% identity to the nucleic acid sequence set forth in SEQ ID NO: 3, or the nucleic acid sequence encoding the lubricin polypeptide has at least 90% identity to the nucleic acid sequence set forth in SEQ ID NO: 6. In a some embodiments, the nucleic acid encodes a HAS2 polypeptide comprising the amino acid sequence set forth in SEQ ID NO: 2, or the nucleic acid encodes a lubricin polypeptide comprising the amino acid sequence set forth in SEQ ID NO: 7.

In an aspect, the disclosure provides a pharmaceutical composition comprising a recombinant viral vector encoding and expressing in vivo in a mammalian host HAS or lubricin, and optionally one or more pharmaceutically acceptable carrier, excipient, or vehicle.

In another aspect, the disclosure provides a method of treating a mammalian subject suffering from, or at risk of developing, osteoarthritis, comprising, intra-articularly administering to said mammalian subject a therapeutically effective amount of the above-detailed pharmaceutical compositions. In an embodiment, the subject is human or canine.

In another aspect, the disclosure provides an adeno-associated virus (AAV)-based biological delivery and expression system for use in the treatment or prevention of OA in human or mammalian joints. In some embodiments, the method is accomplished by long-term gene expression of human or mammalian HAS2 or lubricin in synovial and/or chondrocyte cells, following delivery of rAAV comprising a nucleic acid sequence encoding human or mammalian HAS2 or lubricin, left and right AAV inverted terminal repeats (L ITR and R ITR), the AAV packaging signal and optionally non-viral, non-coding stuffer nucleic acid sequences. In some embodiments, the expression of the human or mammalian HAS2 or lubricin gene within synovial and/or chondrocyte cells is regulated by an inflammation-inducible promoter, which is located upstream of the reading frame of the nucleic acid sequence encoding for human or mammalian HAS2 or lubricin and which is specifically activated by increased levels of immune stimulatory substances.

In some embodiments, the inflammation-inducible promoter is selected from the following: an NF-KB promoter, an interleukin 6 (II-6) promoter, an interleukin-1 (11-1) promoter, a tumor necrosis factor (TNF) promoter, a cyclooxygenase 2 (COX-2) promoter, a complement factor 3 (C3) promoter, a serum amyloid A3 (SAA3) promoter, a macrophage inflammatory protein-1a (MIP-1a) promoter and hybrid constructs thereof. In some embodiments, the rAAV vector genome comprises a nucleic acid sequence set forth in SEQ ID NO: 3, SEQ ID NO: 6, or a biologically effective variant thereof. In some embodiments, the AAV system comprises nucleic acid encoding a marker gene that allows monitoring of the vector genome in the synovial and chondrocyte cells. In some embodiments, the vector comprises a nucleic acid sequence set forth in SEQ ID NO: 3, SEQ ID NO: 6 or a conserved sequence thereof encoding for the same amino acids. In some embodiments, the rAAV vector genome comprises nucleic acid encoding the HAS2 polypeptide set forth in SEQ ID NO: 2 or the lubricin polypeptide set forth in SEQ ID NO: 7. The rAAV vector genome may comprise a nucleic acid molecule having at least 80% or 90% sequence identity with the nucleic acid sequence set forth in SEQ ID NO: 3. In other embodiments, the rAAV vector genome comprises a nucleic acid molecule having at least 80% or 90% sequence identity with the nucleic acid sequence set forth in SEQ ID NO: 6.

In some embodiments of the AAV system, the system comprises a nucleic acid sequence encoding for human or mammalian HAS2 or lubricin, left and right AAV inverted terminal repeats (L ITR and R ITR), a packaging signal and optionally non-viral, non-coding stuffer nucleic acid sequences, wherein the expression of the human or mammalian HAS2 or lubricin gene within synovial and/or chondrocyte cells is regulated by an inflammation-inducible promoter, which is specifically activated by increased levels of immune stimulatory substances, for the treatment or prevention of osteoarthritis (OA).

### Viral Particles and Methods of Producing Viral Particles

Also provided herein are viral particles comprising a nucleic acid encoding a HAS2 or lubricin. Viral vectors can be used for delivery of a nucleic acid encoding a HAS2 or lubricin for expression of the protein in a target cell within a particular location (e.g., a joint). Many species of virus are known, and many have been studied for purposes of delivering nucleic acids to target cells. The exogenous nucleic acid can be inserted into a vector such as an adeno-associated virus (AAV),

In some embodiments, the viral particle is a recombinant AAV particle comprising a nucleic acid comprising one or two AAV ITRs and a sequence encoding a HAS2 or lubricin described herein flanked by one or two ITRs. The nucleic acid is encapsidated in the AAV particle. The AAV particle also comprises capsid proteins. In some embodiments, the nucleic acid comprises operatively linked components in the direction of transcription, control sequences including transcription initiation and termination sequences, and the protein coding sequence(s) of interest (e.g., nucleic acid encoding a fusion protein). These components are flanked on the 5′ and 3′ end by functional AAV ITR sequences. By “functional AAV ITR sequences” it is meant that the ITR sequences function as intended for the rescue, replication and packaging of the AAV virion. See Davidson et al., PNAS, 2000, 97(7)3428-32; Passini et al., J. Virol., 2003, 77(12):7034-40; and Pechan et al., Gene Ther., 2009, 16:10-16, all of which are incorporated herein in their entirety by reference. For practicing some aspects of the invention, the recombinant vectors comprise at least all of the sequences of AAV essential for encapsidation and the physical structures for infection by the rAAV. AAV ITRs for use in the vectors of the invention need not have a wild-type nucleotide sequence (e.g., as described in Kotin, Hum. Gene Ther., 1994, 5:793-801), and may be altered by the insertion, deletion or substitution of nucleotides or the AAV ITRs may be derived from any of several AAV serotypes. More than 40 serotypes of AAV are currently known, and new serotypes and variants of existing serotypes continue to be identified. See Gao et al., PNAS, 2002, 99(18): 11854-6; Gao et al., PNAS, 2003, 100(10):6081-6; and Bossis et al., J. Virol., 2003, 77(12):6799-810. Use of any AAV serotype is considered within the scope of the present invention. In some embodiments, a rAAV vector is a vector derived from an AAV serotype, including without limitation, AAV1, AAV2, AAV3, AAV4, AAV5, AA6, AAV7, AAV8, AAV9, AAVrh.8, and AAVrh.10. In some embodiments, the nucleic acid in the AAV comprises an ITR of AAV1, AAV2, AAV3, AAV4, AAV5, AAV6, AAV7, AAV8, AAV9, AAVrh.8, or AAVrh.10. In further embodiments, the rAAV particle comprises capsid proteins of AAV1, AAV2, AAV3, AAV4, AAV5, AA6, AAV7, AAV8, AAV9, AAVrh.8, or AAVrh.10.

Different AAV serotypes are used to optimize transduction of particular target cells or to target specific cell types within a particular target (e.g., a joint). A rAAV particle can comprise viral proteins and viral nucleic acids of the same serotype or a mixed serotype. For example, a rAAV particle can comprise AAV2 capsid proteins and at least one AAV2 ITR or it can comprise AAV2 capsid proteins and at least one AAV5 ITR. In another example, a rAAV particle can comprise AAV5 capsid proteins and at least one AAV2 ITR. Any combination of AAV serotypes for production of a rAAV particle is provided herein as if each combination had been expressly stated herein.

The rAAV particles can be produced using methods know in the art. See, e.g., U.S. Pat. Nos. 6,566,118, 6,989,264, 6,995,006. In practicing the invention, host cells for producing rAAV particles include mammalian cells, insect cells, plant cells, microorganisms and yeast. Host cells can also be packaging cells in which the AAV rep and cap genes are stably maintained in the host cell or producer cells in which the AAV vector genome is stably maintained. Exemplary packaging and producer cells are derived from 293, A549 or Hela cells. AAV vectors are purified and formulated using standard techniques known in the art.

In some aspects, a method is provided for producing any rAAV particle as disclosed herein comprising (a) culturing a host cell under a condition that rAAV particles are produced, wherein the host cell comprises (i) one or more AAV package genes, wherein each said AAV packaging gene encodes an AAV replication or encapsidation protein; (ii) an rAAV pro-vector comprising a nucleic acid encoding any fusion protein disclosed herein flanked by at least one AAV ITR, and (iii) an AAV helper function; and (b) recovering the rAAV particles produced by the host cell. In some embodiments, said at least one AAV ITR is selected from the group consisting of AAV1, AAV2, AAV3, AAV4, AAV5, AA6, AAV7, AAV8, AAV9, AAVrh.8, and AAVrh.10 ITR. In some embodiments, said encapsidation protein is selected from the group consisting of AAV1, AAV2, AAV3, AAV4, AAV5, AA6, AAV7, AAV8, AAV9, AAVrh.8, and AAVrh.10 capsid protein. In a further embodiment, the rAAV particles are purified. The term “purified” as used herein includes a preparation of rAAV particles devoid of at least some of the other components that may also be present where the rAAV particles naturally occur or are initially prepared from. Thus, for example, isolated rAAV particles may be prepared using a purification technique to enrich it from a source mixture, such as a culture lysate or production culture supernatant. Enrichment can be measured in a variety of ways, such as, for example, by the proportion of DNase-resistant particles (DRPs) present in a solution, or by infectivity, or it can be measured in relation to a second, potentially interfering substance present in the source mixture, such as contaminants, including production culture contaminants or in-process contaminants, including helper virus, media components, and the like.

Also provided herein are pharmaceutical compositions comprising a rAAV particle comprising a nucleic acid encoding HAS2 or lubricin of the invention and a pharmaceutically acceptable carrier. The pharmaceutical compositions may be suitable for a variety of modes of administration described herein, including for example systemic or localized administration. A pharmaceutical composition of a rAAV comprising a nucleic acid encoding HAS2 or lubricin described herein can be introduced systemically, e.g., by intravenous injection, by catheter, see U.S. Pat. No. 5,328,470, or by stereotactic injection, Chen et al., 1994, PNAS, 91: 3054-3057. In some embodiments, the pharmaceutical compositions comprising a rAAV described herein and a pharmaceutically acceptable carrier is suitable for administration to human. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. The pharmaceutical composition may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms. The compositions are generally formulated as sterile and substantially isotonic solution.

### REFERENCES

All publications, patents, and patent applications cited herein are hereby incorporated by reference in their entirety for all purposes.
Aalbers C J et al. 2015. Preclinical potency and biodistribution studies of an AAV5 vector expressing human interferon-b (ARTI02) for local treatment of patients with rheumatoid arthritis. PLOS One 2015, 10:e130612.
Ai, M et al. Anti-lubricin monoclonal antibodies created using lubricin-knockout mice immunodetect lubricin in several species and in patients with healthy and diseased joints. PLOS 2015. 10:e0116237
Apparailly F et al. Adeno-associated virus pseudotype 5 vector improves gene transfer in arthritic joints. Hum Gene Ther 2005; 16: 426-434.
Asokan A, Smulski R. J. 2012. The AAV vector toolkit: poised at the clinical crossroads. Molecular Therapy 20:699-708.
Blewis M E et al. 2007. A model of synovial fluid lubricant composition in normal and injured joints. European Cells and Materials 13:26-39.
Calcedo R et al. 2015. Preexisting neutralizing antibodies to adeno-associated virus capsids in large animals other than monkeys may confound in vivo gene therapy studies. Human Gene Therapy Methods 26:103-105.
Clark, K R et al. (1999). Highly purified recombinant adeno-associated virus vectors are biologically active and free of detectable helper and wild-type viruses Hum Gene Ther 10: 1031-1039.
David-Raoudi M et al. Chondroitin sulfate increases hyaluronan production by human synoviocytes through differential regulation of hyaluronan synthases. Arthritis & Rheumatism 2009, 60:760-770.
Elsaid K A et al. Decreased lubricin concentrations and markers of inflammation in the synovial fluid of patients with anterior cruciate ligament injury. Arthritis & Rheumatism 2008, 58:1707-1715.
Evans C H et al. 2009. Gene therapy of the rheumatic diseases: 1998 to 2008. Arthritis Research Therapy 11:209
Evans C H et al. 2009. Progress and Prospects: genetic treatments for disorders of bones and joints. Gene Therapy 16:944-952.
Flannery C R et al. Prevention of cartilage degeneration in a model of osteoarthritis by intraarticular treatment with recombinant lubricin. Arthritis & Rheumatism 2009, 60:840-847.
Flannery et al. 1999: Articular cartilage superficial zone protein (SZP) is homologous to megakaryocyte stimulating factor precursor and is a multifunctional proteoglycan with potential growth-promoting, cytoprotective, and lubrication properties in cartilage metabolism. Biochem Biophys Res Comm, 254:535-41
Goodrich L R et al. 2013. Optimization of scAAVIL-ira in vitro and in vivo to deliver high levels of therapeutic protein for treatment of osteoarthritis. Molecular Therapy-Nucleic Acids 2:e70.
Guo N et al. A rapid increase in hyaluronan synthase-2 mRNA initiates secretion of hyaluronan by corneal keratocytes in response to transforming growth factor beta. J Biol Chem 2007, 282:12475-83.
Hemphill D D et al. 2014. Adeno-associated viral vectors show serotype specific transduction of equine joint tissue explants and cultured monolayers. Scientific Reports 4:5861-5868.
Hunter D J, Matthews G. Emerging drugs for osteoarthritis. 2011. Expert Opin Emerg Drugs 16:479-491.
Hurlbut G D et al. 2010. Preexisting immunity and low expression in primates highlight translational challenges for liver-directed AAV8-mediated gene therapy. Molecular Therapy 18:1983-1994.
Hyc A et al. Pro- and anti-inflammatory cytokines increase hyaluronan production by rat synovial membrane in vitro. Intern J Molec Medicine 2009, 24:579-585.
Itano N et al. 1999. Three isoforms of mammalian hyaluronan synthases have distinct enzymatic properties. JBC 1999, 274:25086-92.
Itano N et al. 1999. Three isoforms of mammalian hyaluronan synthases have distinct enzymatic properties. JBC 1999, 274:25086-92.
Keiser N W et al., Engelhardt. 2011. Unique characteristics of AAV1, 2, and 5 viral entry, intracellular trafficking and nuclear import define transduction efficiency in Hela cells. Hum Gene Ther 22:1433-1444.
Kwiecinski et al. 2011: The effect of molecular weight on hyaluronan's cartilage boundary lubricating ability—alone and in combination with proteoglycan 4. Osteoarthritis Cartilage 19:1356-62.
Kyostio-Moore S et al. 2015. Over-expression of cystatin C in synovium does not reduce synovitis or cartilage degradation in established osteoarthritis. Arthritis Res Ther 17:5-21.
S. Kyostio-Moore et al. Local gene delivery of heme oxygenase-1 by adeno-associated virus into osteoarthritic mouse joints exhibiting synovial oxidative stress. Osteoarthritis and Cartilage Volume 21, Issue 2, February 2013, Pages 358-367.
Lee H H et al. Persistence, localization, and external control of transgene expression after single injection of adeno-associated virus into injured joints. Hum Gene Ther 2013, 24:457-466.
Li P et al. Hylan G-F 20 maintains cartilage integrity and decreases osteophyte formation in osteoarthritis through both anabolic and anti-catabolic mechanisms. Osteoarthritis Cartilage 2012, 20:1336-46.
Loeser R F. Osteoarthritis year in review 2013: biology. Osteoarthritis and Cartilage, 21:1436-1442.
Mease P. J et al. 2009. Local delivery of a recombinant adeno-associated vector containing a tumor necrosis factor alpha antagonist gene in inflammatory arthritis: a Phase 1 dose-escalation safety and tolerability study. Ann Rheum Dis 68:1247-1254.
Mietzsch M et al. 2014. Differential adeno-associated virus serotype-specific interaction patterns with synthetic heparins and other glycans. J Virology 88:2992-3003.
Miltner O et al. Efficacy of intraarticular hyaluronic acid in patients with osteoarthritis—a prospective clinical study. Osteoarthritis Cartilage 2002, 10:680-6.
Mingozzi F et al. 2013. Prevalence and pharmacological modulation of humoral immunity to AAV vectors in gene transfer to synovial tissue. Gene Therapy 20:417-424.
Momberger T S et al. Hyaluronan secretion by synoviocytes is mechanosensitive. Matrix Biology 2005, 24:510-519.
Nishida Y et al. Antisense inhibition of hyaluronan synthase-2 in human articular chondrocytes inhibits proteoglycan retention and matrix assembly. JBC 1999, 274:1893-21899.
Ortved K F et al. Implantation of rAAV5-IGF-I transduced autologous chondrocytes improves cartilage repair in full-thickness defects in the equine model. Mol Ther 2015, 23:363-373.
Payne K A et al. Single intra-articular injection of adeno-associated virus results in stable and controllable in vivo transgene expression in normal rat knees. Osteoarthritis Cartilage 2011, 19:1058-1065.
Plickert H D et al. Hyaluronic acid concentrations in synovial fluid of dogs with different stages of osteoarthritis. Research in Veterinary Science 2013, 94:728-734.
Rapti K et al. 2012. Neutralizing antibodies against AAV serotypes 1, 2, 6, and 9 in sera of commonly used animal models. Molecular Therapy 20:73-83.
Rhee D K et al. Consequences of disease-causing mutations on lubricin protein synthesis, secretion, and post-translational processing. JBC 2005, 280:3125-3132.
Ryan M Z C et al. Proteoglycan 4 expression protects against the development of osteoarthritis. Sci Transl Med 2013, 5:176ra34.
Sarzi-Puttini P et al. Osteoarthritis: an overview of the disease and its treatment strategies. Semin. Arthritis Rheum 2005, 35:1-10.
Schmid T et al. 2001. Superficial zone protein (SZP) is an abundant glycoprotein in human synovial fluid and serum. Trans Orthop Res Soc 26:82.
Sharkey M. The challenges of assessing osteoarthritis and postoperative pain in dogs. 2013. The AAPS Journal 15:598-607
Vugmeyster Y et al. Disposition of human recombinant lubricin in naïve rats and in a rat model of post-traumatic arthritis after intra-articular or intravenous administration. AAPS J 2012, 14:97-104.
Waller K A et al. Role of lubricin and boundary lubrication in the prevention of chondrocyte apoptosis. PNAS 2013.
Watanabe K and Yamaguchi Y. Molecular identification of a putative human hyaluronan synthase. JBC 1996, 271:22945-48
Watanabe, S et al. Adeno-associated virus mediates long-term gene transfer and delivery of chondroprotective IL-4 to murine synovium. Molecular Ther 2000; 2: 147-151.
Watson R S et al. scAAV-mediated gene transfer of interleukin-1-receptor antagonist to synovium and articular cartilage in large mammalian joints. Gene Therapy 20:670-677.
Yoshida M et al. Expression analysis of three isoforms of hyaluronan synthase and hyaluronidase in the synovium of knees in osteoarthritis and rheumatoid arthritis by quantitative real-time reverse transcriptase polymerase chain reaction. Arthritis Research Therapy 2004, 6:R514-R520.

### EXAMPLES

### Example 1—HAS2 AAV Vector Construction and Evaluation

### Example 1a—Overview

rAAV vectors containing codon-optimized canine HAS2 cDNA and a ubiquitous promoter were generated and packaged into AAV2 or AAV5 capsids. Large-scale vector lots were generated by triple transfection method and purified by CsCl gradient. Vector yields were quantitated by qPCR to the bovine growth hormone (BGH) polyA site (pA). A total of 4 lots were generated for AAV2/HAS2 (of which 3 were pooled for in vivo studies, 2×10¹³ DRP/total). Two lots were generated for AAV5/HAS2 (5×10¹² DRP/total) to test consistency of production yields and obtain sufficient amount of virus.

Twenty-two adult healthy dogs, seronegative for AAV2 and AAV5 capsids, received rAAV2 (1, 5 and 10×10¹¹ vg/joint), rAAV5 (5×10¹¹ vg/joint) or PBS (control) via intra-articular injection. No adverse clinical signs were observed following the 28-day study. Histopathological analysis showed minimal synovial inflammation in joints treated with rAAV5 and no significant changes in the rAAV2 treatment groups. Vector genomes (VG) were detected in the synovium of all the rAAV-treated joints and in the majority of cartilage samples. The rAAV5 vectors resulted in higher VG detection and mRNA expression compared to rAAV2 in both tissues. A trend of increased HA levels in the synovial fluid of the treated joints was noted. In summary, the disclosed results demonstrated gene transfer to canine joint tissues and an acceptable safety profile with rAAV2 and rAAV5 vectors encoding HAS2 when administered by single intra-articular injection to canines.

### Example 1b—Methods

### Cloning and Generation of HA Expression Vector.

Canine HAS2 gene (GenBank XM 539153.3; SEQ ID NO: 1) was codon-optimized for expression in canines by algorithm from GeneArt/Invitrogen. The codon-optimized canine HAS2 cDNA (1656 bp; SEQ ID NO:3) was synthesized with flanking NheI-NsiI restriction enzyme sites This fragment was then cloned into a plasmid containing ubiquitous chicken b-actin promoter (CBA), a hybrid intron and a bovine growth hormone (BGH) polyA (pA). The resulting pCBA-HI-cHAS2-BGHpA plasmid was purified using maxi kit (Qiagen) for expression analyses.

Expression of cHAS2 In Vitro and Production of HA.

A plasmid vector containing cHAS2 was transfected into 293 cells and the conditioned media and cell lysates were collected into 250 μl of RIPA buffer plus protease inhibitors 3 days later. Cell lysates were spun to remove cell debris and 30 μl of the cell lysate was loaded on a 4-12% nu-page gel and run in 1× Mops buffer. The protein gel was transferred to a nitrocellulose membrane and probed with anti-HAS2 (sc-34-068; Santa Cruz Biotechnology) in 5% milk in PBS-T 0.1% tween-20 overnight at 4ºC. A donkey anti-goat secondary antibody (at 1:5000 dilution) was used as secondary antibody. Beta actin detection was used to show equal loading of cell lysates.

### Quantitation of HA Levels and Molecular Weight In Vitro Cultures.

The production of HA by HAS2 expressing cells was evaluated by transfecting pCBA-HI-cHAS2-BGHpA into 293 cells (in Optimem or complete medium). The conditioned media was quantitated for HA levels using HA test kit (Corgenix, Inc.). This kit contains a HA-binding protein derived from aggrecan. The molecular weight of HA was assessed by running concentrated conditioned media on agarose gel. Various HA size markers were run in parallel (Select-HA HiLadder, Hyalose, Austin, TX). A similar gel was run in parallel followed by digestion of hyaluronidase for 24 h. Both gels were stained with All-stain.

Generation of rAAV Vector with cHAS.

The cHAS2 expression cassette was cloned into a AAV ITR-containing plasmid to generate expression cassette flanked by AAV inverted terminal repeats (pre-viral plasmid pDC627) to construct psITR/CBA-HI-cHAS2-BGHpA. A 600 bp stuffer DNA (chromosome 16 P1 clone 96.4B) was included upstream of expression cassette to generate viral vector genome of 4500 bp total. To test packaging of the cHAS2 expression cassette containing plasmid, 293 cells were seeded at 8×10⁵ cells/well (6-well plates) and the following day transfected with psITR/CBA-HI-cHAS2-BGHpA, or psp70/EGFP, pHLP-19cap2 or p5repCMVcap5 plasmids and pAdHELP in duplicates (Promega CaPO₄ kit). Cells were collected 3 days later and the lysates were titered for BGHpA copies using qPCR analysis and primer/probe to BGHpA sequences (SEQ ID NOS:12-14). A plasmid containing BGHpA was used as a standard. The rAAV virus yields were expressed as amount of DNase resistant particles (DRP) per cell. Large scale vector production was performed using triple transfection of psITR/CBA-HI-cHAS2-BGH, pIM45BD rep-cap plasmid for AAV2 vectors and pHLP19-cap5 for AAV5 vectors, and pAdHELP. The vector was purified by CsCl and resulting vector lot titered using TaqMan analysis and primer/probe to BGHpA sequences (Applied Biosystems/Life Technologies).

Efficacy of rAAV/cHAS2 in Rabbit Chondrocytes and Synoviocytes In Vitro.

The ability of the vector to transduce joint cell types such as primary synoviocytes and chondrocytes was tested using rabbit cells. The cells were infected with 1e5 DRP/cell and cultured for 3 days. The cell lysates were collected for HAS2 protein detection by Western blot and culture media was quantitated for HA levels as described above. To test the effect of HA production on matrix degrading proteases, inflammatory cytokines and cartilage structural protein production in disease conditions, the cells were first infected with rAAV vectors followed by IL-1b stimulation 24 h later. After 24 h, both cells and culture media were collected for mRNA analysis and HA production.

rAAV/cHAS2 Evaluation in Normal Canine Joints.

Mixed breed dogs were used (male and females, 8-10 kg). Canines with serum titer of <4 or 4 to AAV2 and/or AAV5 capsids were used for the study. rAAV2 and AAV5 vectors encoding for cHAS2 were administered (AAV2: 1, 5 and 10×10¹¹, AAV5, 5×10¹¹ DRP/joint) by intra-articular route. PBS was used as negative control. Animals were observed for clinical signs (pain, lameness, swelling of the injected joint and other abnormalities) once daily for 7 days prior to injections, twice daily for 7 days after injection and then once daily for the duration of the study. Animals were sacrificed 4 weeks later. Whole blood samples were collected −7, 1, 14 and 28 days after vector administration for white blood cell (WBC) counts. Synovial fluid (SF) samples were collected on days −7, day 14 and day 28 for quantitation of HA levels. Synovial tissue, cartilage and liver samples were collected for DNA and RNA isolation. cHAS2 vector genome and mRNA copies were determined by qPCR analysis using BGHpA primer/probe sets (Applied Biosystems/Life Technologies). For histological analysis, the medial side of the knee (tibia, femur, synovium) was embedded in paraffin and sectioned. Sections were stained with toluidine blue and examined by a board certified veterinary pathologists. Cartilage was evaluated for severity of cartilage lesions and proteoglycan loss (scoring: 0-5). Synovial pathology was scored for density of inflammatory cells (scoring: 0-5) as no synovial thickening was observed.

### Example 1c—Results

### Codon-Optimization and Generation of HAS2 Expression Cassette.

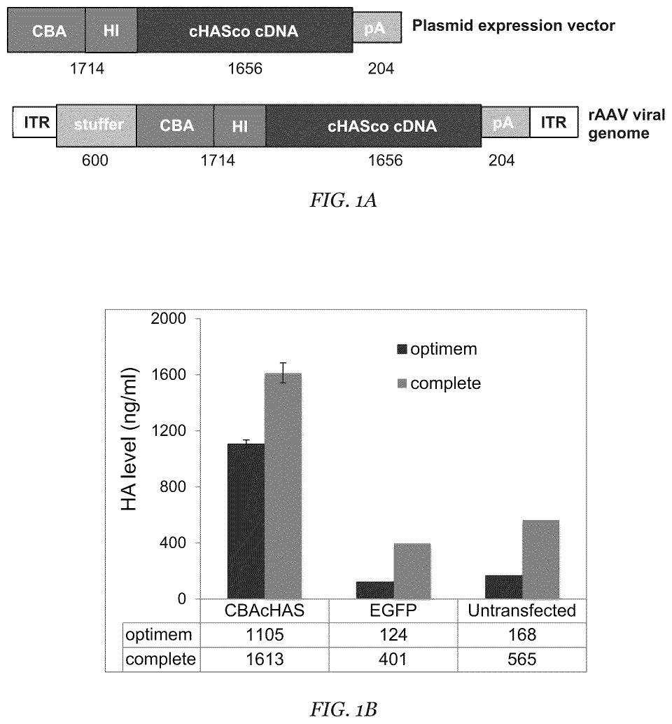

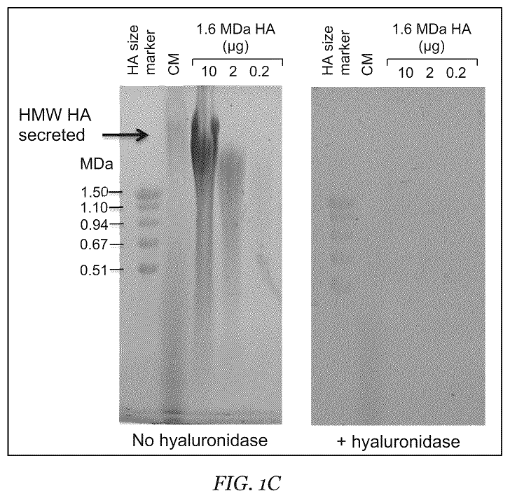

Mammalian HAS2 is a highly conserved protein. For example, the human and canine amino acid sequence for HAS2 contains only 2 amino acid differences (99.3% identity). Similarly, only 3 amino acids are different between canine and rabbit HAS2 (99.5%). At the DNA level, the similarity between canine and human HAS2 synthase cDNA is 93.9%. As codon-optimization can improve by gene expression, the canine HAS2 GenBank sequence (XM 539153.3) was optimized by GeneArt/Invitrogen. This resulted in a nucleotide sequence having 78% similarly to the original GenBank sequence. The GC content of the optimized cDNA was increased from 44.4% to 59.0%. This cDNA was used to generate ubiquitous expression plasmid with a CBA promoter to allow constitutive expression of HAS2 unlike to endogenous promoter (FIG. 1A). The CBA promoter is less influenced by various pro- and anti-inflammatory cytokines.

### HAS2 Expression and HA Production In Vitro.

To test the expression of HAS2 protein in vitro, 293 cells were transfected with two clones (#1 and 2) of CBA-HI-cHAS2-BGHpA plasmid vectors followed by analysis of cell lysates for HAS2 protein (a membrane protein) by Western blot. Cells transfected with the expression plasmid showed a band at 64 kDa that is the expected size of cHAS (not shown). We next evaluated whether over-expression of HAS2 protein in 293 cells resulted in increased detection of HA in the culture media indicating both production and secretion of HA across the cell membrane. The HA levels in media from cells transfected with pCBA-cHAS2 were increased by 6.5- and 9-fold compared to untransfected and CBA-EGFP transfected cells, respectively (FIG. 1B). The data thus confirmed that over-expression of cHAS2 in cells resulted in increased HA levels in the extracellular compartment. The size of HA produced in vitro was evaluated on agarose gel. The data showed a high molecular weight HA in the conditioned media obtained from cells transfected with HAS2 expression cassette. The size of this material was larger than 1.5 Mega Dalton (MDa) (based on estimation with HA molecular weight markers). This material disappeared after digestion with hyaluronidase indicating material was HA (FIG. 1C).

Generation of rAAV Vectors with HAS2 Expression Cassette.

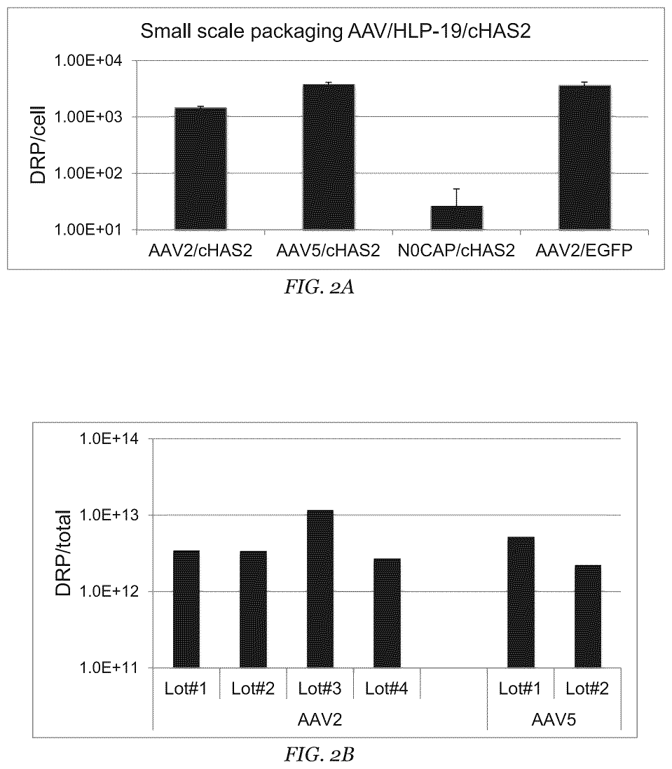

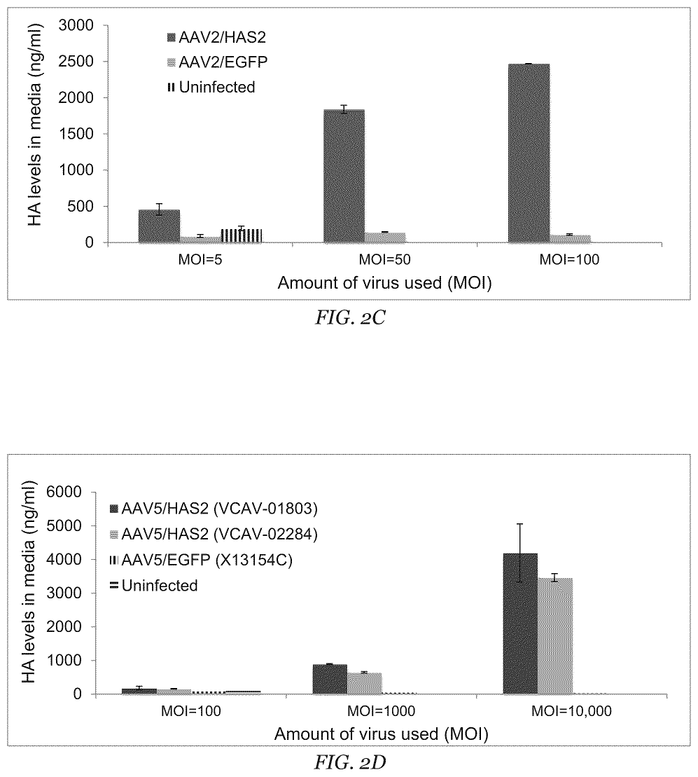

The cHAS2co expression cassette was subsequently cloned into plasmid with AAV ITRs. A schematic of the resulting viral genome is shown in FIG. 1A. The ability to generate rAAV vectors with AAV2 and AAV5 capsids and HAS2 cDNA was tested in a small-scale packaging experiment (FIG. 2A) followed by larger scale vector production. Both AAV2 and AAV5 vectors could be generated using standard triple transfection methods (FIG. 2B). The potency of this material was tested by infecting 293 cells and analyzing production of HA levels in the culture media. Both AAV2 and AAV5 vectors resulted in dose-responsive increase of HA in the culture media (FIGS. 2C, D).

rAAV/HAS2 Vector Evaluation+ in Normal Canine Joint.

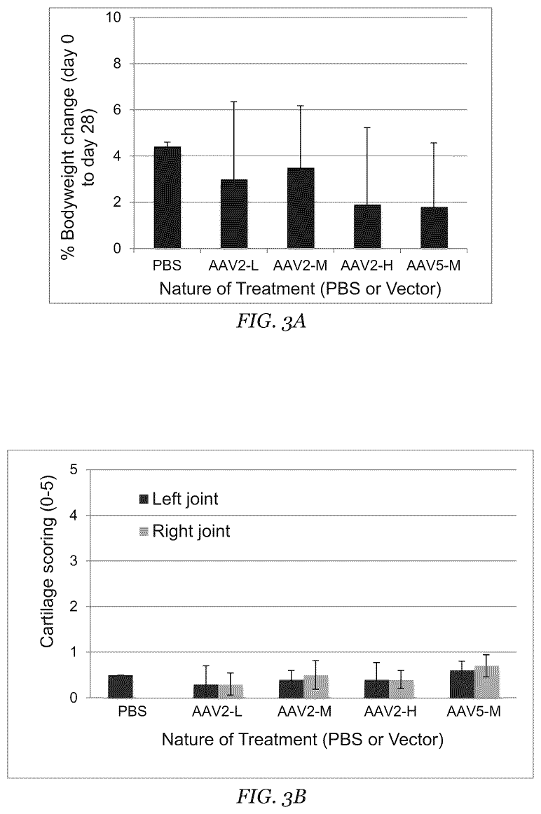

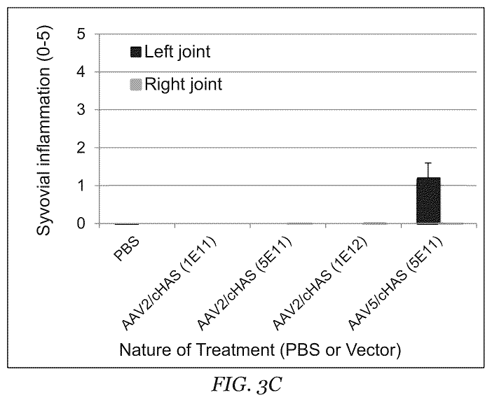

rAAV2 and AAV5 vectors with cHAS2 were delivered into joints of normal dogs by intra-articular administration and the animals were evaluated for 28 days. No adverse clinical signs, bodyweight changes (FIG. 3A), lameness or death were observed during the study. Some animals had elevated white blood cell (WBC) counts on day −7 potentially due to stress of shipping. In general, WBC counts on Days 1, 14 and 28 were within normal limits. Histological evaluation of knees from PBS-, AAV-injected (left) and contralateral (un-injected) showed very minimal proteoglycan loss and cartilage degeneration (score range 0-0.5; maximum score 5) (FIG. 3B). These minimal changes were typical age-related spontaneous changes. Minor synovial changes were observed for PBS- and AAV2-treated and contralateral joints (FIG. 3C). Minimal to mild synovitis (generally extended into joint capsule and medial collateral ligament) were seen in all left knees of males and females treated with AAV5 vector (no synovitis observed in the contralateral joint). Thus, overall the treatment was well tolerated with little adverse effect observed.

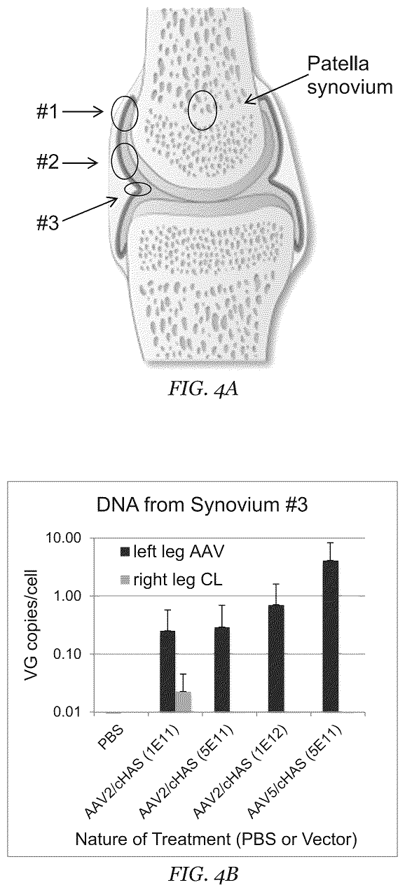

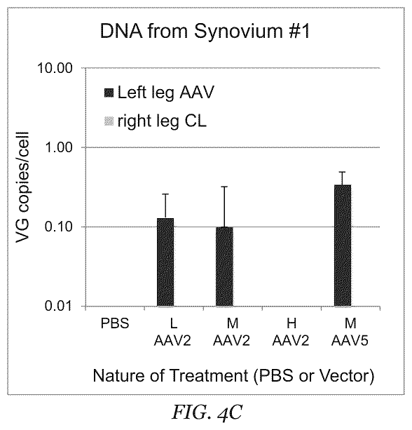

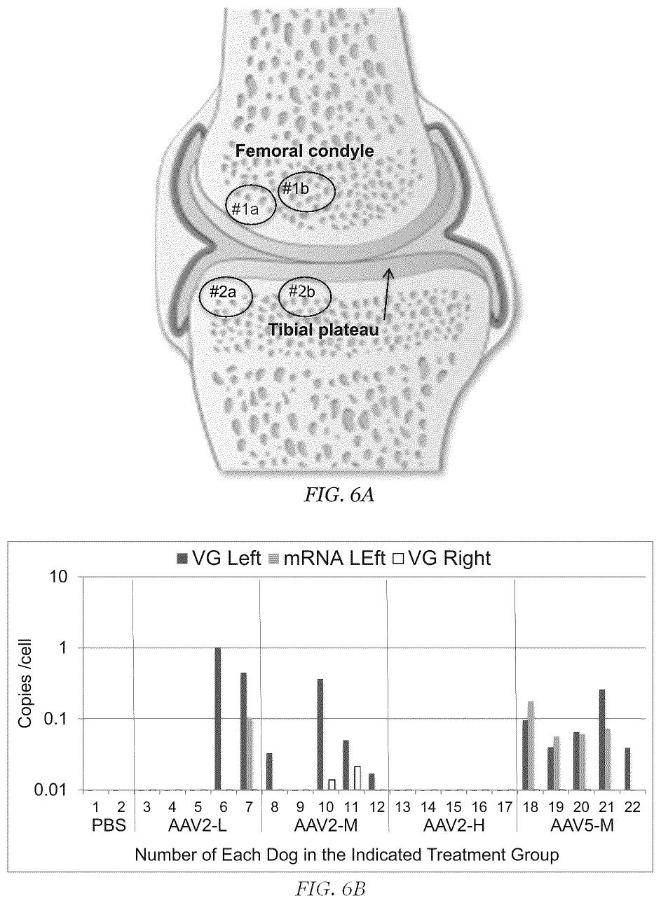

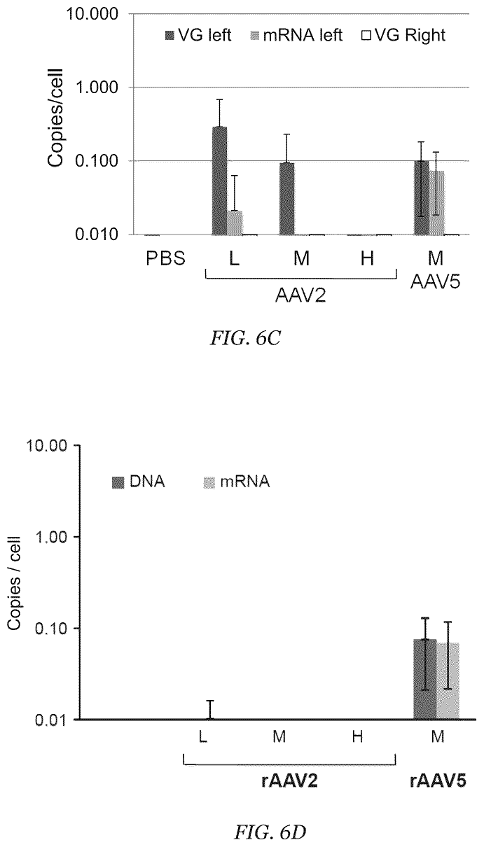

Tissue samples collected from synovium and cartilage were analyzed for detection of viral genomes (FIGS. 4A, 6A). Synovial samples collected closest to the injection site, sample #3, showed the presence of vector genomes in all of the AAV-treated joints (FIG. 4B). AAV2-treated joints contained roughly 0.01 to 2 vector genomes (VG)/cell. Interestingly, minor dose response was observed with AAV2 despite 10-fold difference between the low and high dose groups. Joints treated with AAV5 vectors showed higher and a more consistent detection with a range of 1 to 12 copies/cell. In some contralateral (un-injected) joints a low level of VGs was detected which was more pronounced in the low AAV2-treatment group and more sporadic in higher AAV2 doses and AAV5 groups (not shown).

Synovium sample collected further up from the injection site, sample #1, was analyzed to evaluate AAV spread in the joint (FIG. 4C). Joints injected with AAV2 low dose showed a more consistent detection of VGs. These levels were comparable to those measured in synovium sample #3. In the AAV5 treatment group, all synovium #1 samples had consistently detectable VGs (within 3-fold). These, however, were lower than VG levels detected in synovium #3 therefore demonstration location dependent transduction.

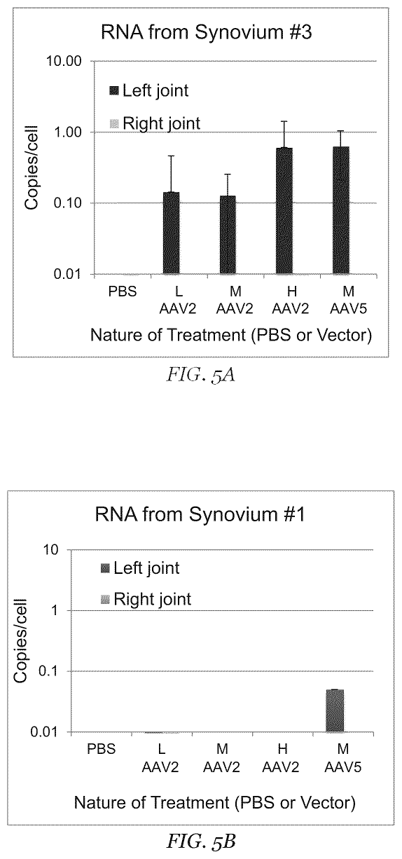

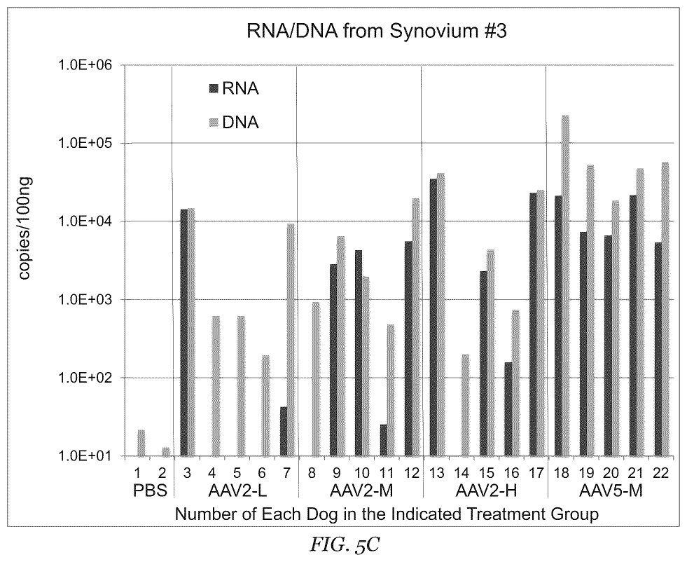

Expression from vector genome was analyzed by quantitating vector derived mRNA. For synovial sample #3, expression was detected in ⅖, ⅘ and ⅘ of the AAV2-treated low, medium and high groups while all AAV5 treated joints had detectable mRNA copies (FIG. 5A). Vector expression was also detected for AAV5 vectors in synovium #1 though the levels were lower similar to reduced detection of VGs at this location (FIG. 5B). The detection of mRNA correlated well with VG detection; the mRNA and VG DNA in each individual injected joint in synovium sample #3 is shown as an example (FIG. 5C).

### Vector Genome Detection in Canine Cartilage.

Cartilage samples collected from femoral condyles and tibial plateaus were analyzed for detection of viral genomes (VGs; FIG. 6A). Vector DNA and mRNA detected in each individual injected joint and a group average in femoral condyles is shown as an example (FIG. 6B, C). The data showed that the AAV5 vector was present in a consistent manner in cartilage and showed comparable levels of vector derived transcripts. A comparable dose of AAV2 vector (medium) resulted in similar VG levels as AAV5 vector but showed approximately 100-fold lower mRNA levels. Additionally, AAV2 VG copies appeared to have a reverse correlation to vector dose. rAAV5-injected joints also showed vector detection and expression in cartilage samples collected from the tibial plateau while none was detected in rAAV2-treated joints (FIG. 6D) All vectors resulted in minimal vector DNA detection in the contralateral (un-injected) joints.

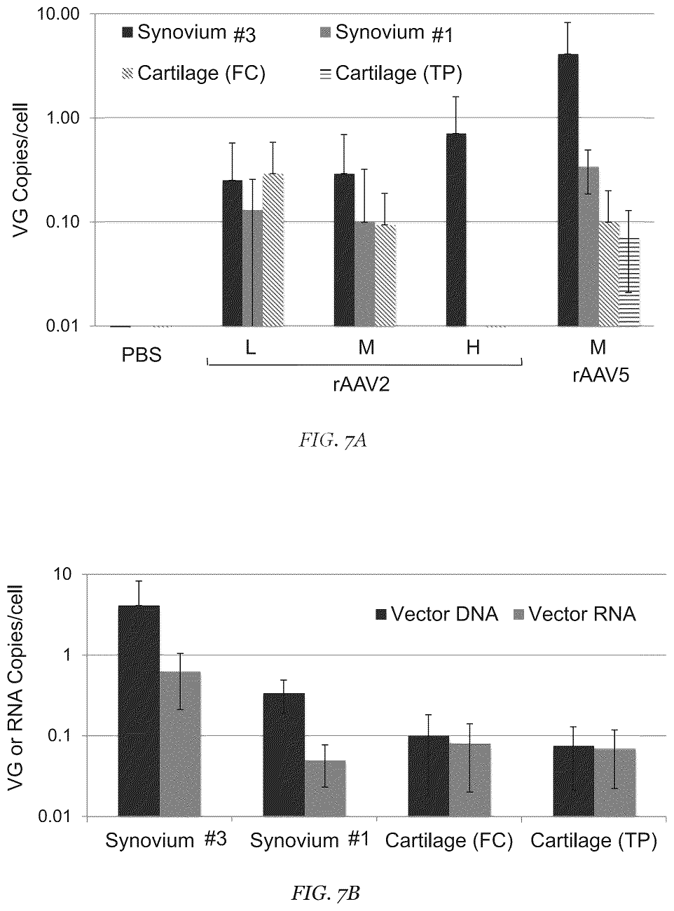

The synovial and cartilage results are summarized in FIG. 7A. For synovial gene transfer, AAV5 vectors resulted in approximately 10-fold higher vector DNA copies in both synovial sample locations compared to that of AAV2 vector. Gene transfer to cartilage was 10 to 20-fold lower than that of synovium by AAV5, while AAV2 vector genomes were observed at similar levels both in the synovium and cartilage. The rAAV5 vector derived genome and mRNA detection are summarized in FIG. 7B showing consistent gene transfer and expression by rAAV5/HAS2 vector in all tissue samples examined. Applicants deem this result to be highly unexpected.

### Analysis of HA Levels in Synovial Fluids.

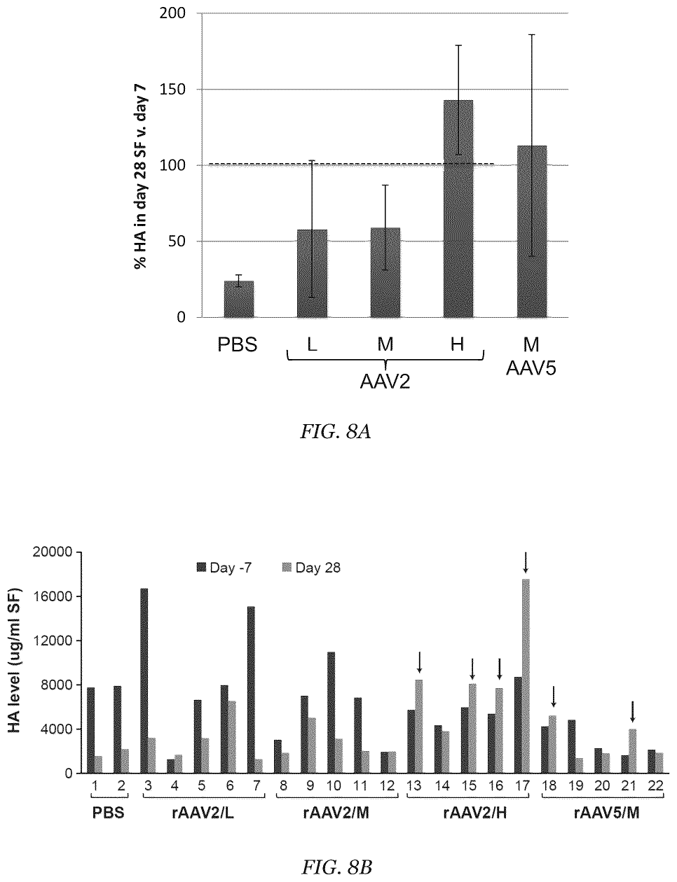

To determine whether any changes in HA levels in synovial fluids could be detected after rAAV vector administration, the synovial HA levels were quantitated in samples collected on days −7 (baseline) and day 28. As high level of variation was detected among the animals, the HA levels in each animal were normalized to baseline levels in each animal. The data showed that compared to PBS-treated animals, both AAV2/high and AAV5/medium doses on average increased HA levels in synovial fluids (FIGS. 8A & 8B).

### Example 1d—Conclusions

To provide overexpression of HA in the joint in vivo, Applicants generated rAAV vectors with two capsid serotypes. The choice of AAV capsid serotype is important as any pre-existing neutralizing antibodies in target species can neutralize the therapeutic vector and hence block gene transfer by rAAV vectors. The results disclosed herein showed that the majority of the dogs analyzed had low levels of neutralizing antibodies to both AAV2 and AAV5 capsids. As such, Applicants tested AAV2 and AAV5 capsid localization directly in the target tissue, namely canine knee joint, after intra-articular injection. Since HA expression is expected to be beneficial both for synoviocytes and chondrocytes, vector genome copies were quantitated in canine synovial and cartilage samples, respectively. The data showed that AAV2 provided very inconsistent gene transfer to canine synovial and cartilage tissues in vivo and showed little dose-response effect, the reasons of which are unclear. Similar experiments performed in rabbit OA joints demonstrated very consistent rAAV2 vector genome detection with a comparable vector dose (Kyostio-Moore 2015). In contrast to AAV2 vector, AAV5 vector genomes were detected in a consistent manner in both tissue types (n=5/group).

Importantly, detection of AAV5 in cartilage samples was surprising and unexpected since cartilage has been reported as difficult to transduce in in vivo conditions, due to extensive extracellular matrix and currently, there are no other reports on detection of AAV5 in cartilage of large animals after intra-articular delivery. In addition, the instantly disclosed canine studies produced unpredictably high levels of rAAV5 vector in canine synovial tissue, which were also about 2-logs higher in synovium compared to that in cartilage, indicating a preference for canine synovial lining by AAV5. This preferential expression pattern likewise could not have been predicted in advance of this disclosure.

In addition to detecting the high level of vector, recombinant HAS2 expression by mRNA analysis was confirmed in canine synovial and cartilage tissues, indicating that the CBA promoter was functional in both tissue types. Further, detection of transcripts by AAV5 in cartilage samples confirmed that chondrocytes were transduced by the vector rather than the virus being sequestered in the extracellular matrix of cartilage. For AAV2, comparable levels of vector genomes and transcripts were also observed in the synovial lining. However, the mRNA expression from AAV2 vectors was surprisingly approximately 100-fold lower than detection of corresponding vector genomes in cartilage samples, suggesting that some of the vector remained outside the chondrocytes, possibly retained to the extracellular matrix. These critical differences could only be appreciated after Applicants conducted significant non-routine experimentation.

Though the vectors were administered only to one joint in each animal, vector genomes were occasionally detected in the contralateral un-injected joint. This was mostly observed in the synovial samples obtained from AAV2-treated joints. However, none of the animals with vector genomes observed in the contralateral joints had any detectable HAS2 transcripts in these joints.

In summary, the data disclosed herein indicate that AAV5 capsid provides good gene transfer via intra-articular delivery to the canine joint. This is based on low pre-existing humoral immunity to AAV5 in subjects and ability to transduce joint tissues after intra-articular injection. Injections into joints can provide gene transfer not only to synovial lining but also to cartilage chondrocytes. Both tissue types will benefit from the increased HA synthesis afforded by the disclosed gene delivery compositions and methods: synovium, by the increased ability to provide lubrication in synovial fluid; and cartilage, by serving as a scaffold for increased matrix attachment and hence improved cartilage health. These results indicate that over-expression of HA by AAV-mediated HAS2 gene transfer to the disease site will decrease OA pathology and pain.

### Example 2—Lubricin AAV Vector Construction and Evaluation

### Example 2a—Overview

Recently, it has been demonstrated that intra-articular injections of recombinant lubricin protein reduced cartilage degeneration in a rat OA model (Flannery 2006). However, recombinant lubricin administered into joints had a very short half-life in the synovial fluids with the majority protein being cleared within 72 h (Vugmeyster 2011). As such, repeated intra-articular injections would be required which is laborious, stressful and costly. In contrast to HAS2 (see Example 1), lubricin is encoded by a large cDNA and contains multiple DNA repeats in its mucin-like domain making it difficult to fit into rAAV vectors and to express in high levels, respectively. To avoid this problem, Applicants generated a shortened canine lubricin cDNA to optimize small expression cassettes for increased lubricin production. Importantly, prior to this disclosure, neither the full-length canine lubricin sequence nor the shortened form as disclosed herein were known.

Briefly, Applicants generated a cDNA for a full-length canine lubricin that was subsequently used to design a shortened and codon-optimized version of canine lubricin (cLubico). The latter was then used to construct various lubricin expressing plasmids. The plasmids were characterized for lubricin mRNA and protein production after transfection into HEK293 cells. The data showed both production of lubricin mRNA and secreted lubricin from each construct. Lastly, Applicants generated rAAV vectors with cLub1 expression cassette and demonstrated the feasibility of rAAV/cLub1 vector production. HEK293 cells infected with this construct synthesized and secreted canine lubricin.

### Example 2b—Methods

### Cloning of Canine Lubricin.

Since no canine full-length lubricin cDNA exists in GenBank (incomplete sequence: GenBank no. ABD38836.1), a complete canine cDNA was obtained from custom synthesized canine cartilage cDNA library. To accomplish this, overlapping fragments were generated using qPCR with various primers. The full-length cDNA (SEQ ID NO:4) was then used to design a shortened form of canine lubricin (cLub1) similar to a published shorter version of human lubricin (Flannery 2009). This canine shorter lubricin contained a deletion in a sequence encoding amino acids 378 to 782. The shortened lubricin sequence was codon-optimized (cLubico) and synthesized (GeneArt/Invitrogen). The cLubco fragment (KpnI blunt to PmeI) was cloned into the Mfe (blunted)—PmeI site of a plasmid containing a CMV enhancer, chicken β-actin promoter and shortened hybrid intron (HIb)(min CBA), and bovine growth hormone (BGH) polyadenylation (pA) site. The ligation reaction was transformed into E. coli Stable II cells and grown at 30° C. to minimize DNA rearrangements. Resulting clones were analyzed by restriction enzyme analyses and the cloning junctions were analyzed by DNA sequencing. Additional constructs were generated that contained 6× histidine (6×His) codons and modifications in two “ATG” sequences present in the intron sequence. Expression plasmids were used for analysis of lubricin expression in vitro.

### Expression Analysis for Canine Lubricin.

Lubricin expression plasmids were transfected into HEK293 cells using Lipofectamine 2000 (Invitrogen) and cells were grown for 72 h. To analyze lubricin mRNA expression, the cells were collected and transcript levels were measured by real-time (RT) qPCR assay using primers/probe specific to BGH pA (7500 Real-Time PCR System; Applied Biosystems, Foster City, CA). For analysis of protein production, the culture media were collected and concentrated approximately 20- to 30-fold (100 k MWCO filter, Millipore). Samples were run on a 4-12% Bis-Tris gel or 3-8% Tris-acetate (NuPAGE; Thermo Fisher Scientific) SDS-PAGE gel (reduced) in MOPS or Tris-acetate buffer, respectively. Lubricin was detected by Western blot using a mouse anti-lubricin antibody (9G3, Millipore) (Ai 2015) and a goat anti-mouse-HRP as a secondary antibody (Jackson ImmunoResearch Laboratories, West Grove, PA).

### Generation of AAV/cLubico.

The cLub1 expression cassette was cloned into a AAV inverted terminal repeats (ITRs) containing plasmid to generate expression cassette flanked by AAV ITRs (previral plasmid pDC627) to construct psITR/minCBA-HI-cLubico-BGHpA. To test the packaging of the cLub1 expression cassette containing plasmid, 293 cells were seeded at 8×10⁵ cells/well (6-well plates) and the following day transfected with psITR/minCBA-HI-cLubico-BGHpA, or psp70/EGFP, pHLP-19cap2 (AAV2) or p5repCMVcap5 (AAV5) plasmids and pAdHELP (Promega CaPO₄ kit) to package vectors into AAV2 or AAV5 capsids. Cells were collected 3 days later and the lysates were quantitated for vector yield by qPCR assay (7500 Real-Time PCR System) using primer/probes specific to BGH pA sequences (Applied Biosystems/Life Technologies) and a standard curve of serially diluted linearized plasmid DNA containing BGH pA. The rAAV virus yields were expressed as amount of DNase-resistant particles (DRP) per cell (Clark 1999).

Research scale vector production was performed using triple transfection of psITR/minCBA-HI-cLubico-BGH, pHLP19-cap5 for AAV5 vectors, and pAdHELP. The vectors were purified by CsCl gradient and yields quantitated as described above (University of Massachusetts Medical School, Worcester, MA).

### Example 2c—Results

### Generation of Short Canine Lubricin.

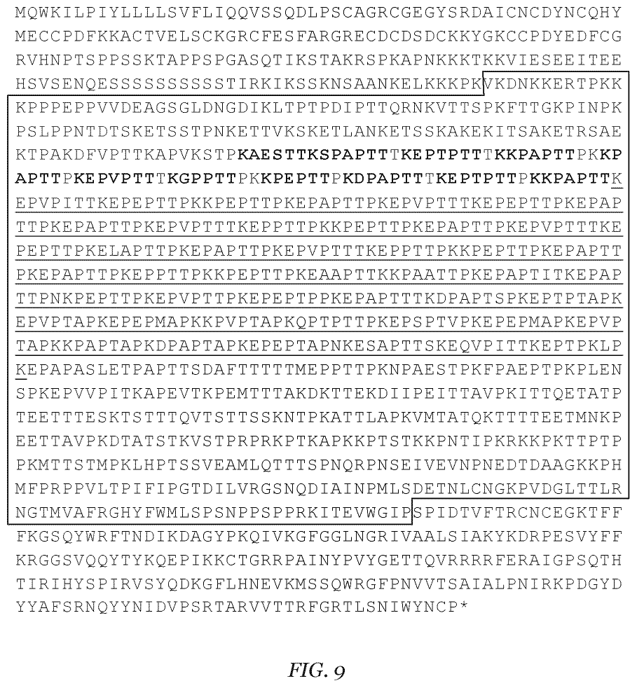

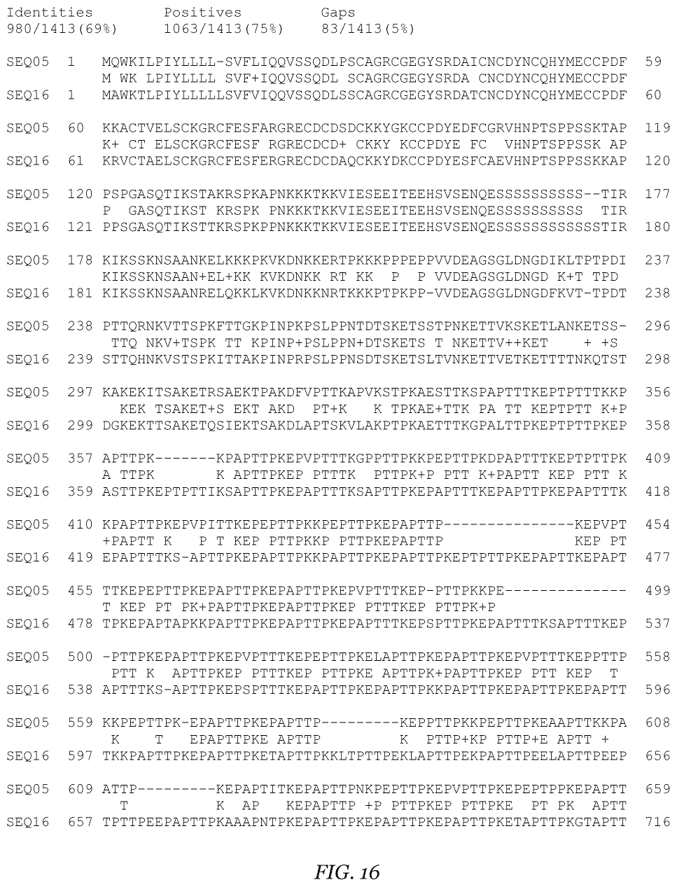

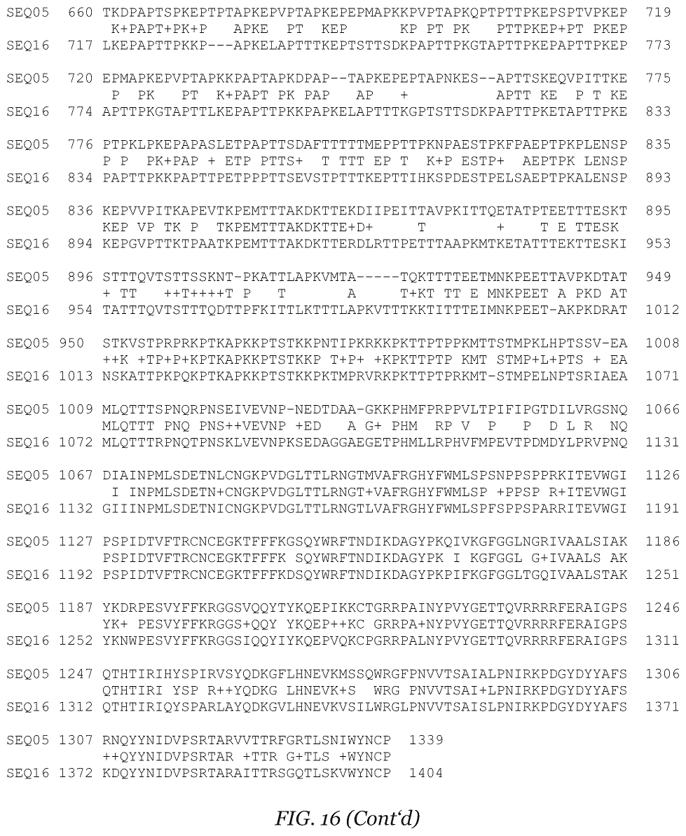

As the canine lubricin sequence present in GenBank was missing a large portion of exon 6 (encoding 857 amino acids), a full-length canine lubricin cDNA (4017 bp, not including the stop codon; SEQ ID NO:4) was generated that encoded for a protein with total of 1339 amino acids (SEQ ID NO: 5; FIG. 9), which is slightly smaller than human sequence of 1404 amino acids). At the amino acid level the canine lubricin sequence had 79% identity to that of human lubricin (SEQ ID NO:11; FIG. 16).

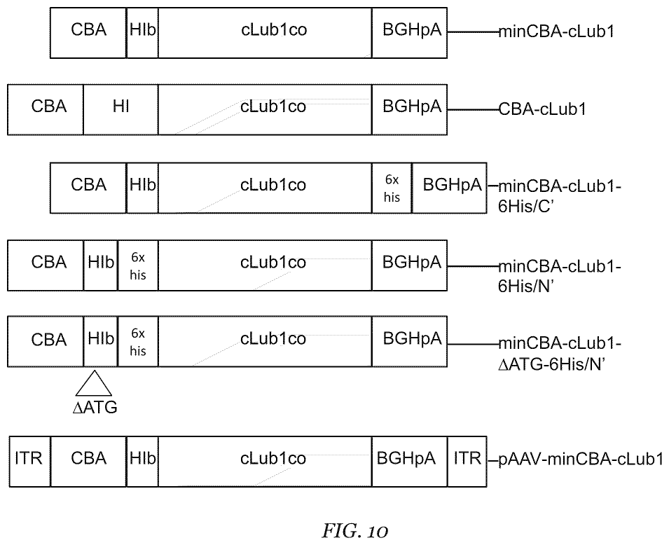

Since the full-length canine lubricin was too large to fit into the rAAV vector due to a packaging limit, a shortened version of canine lubricin was generated. This shorter version of canine lubricin, “Lub1,” was generated by deleting a sequence coding for amino acids 378 to 782 in the mucin-like domain and resulted in a 2949 bp long cDNA (SEQ ID NO: 6) that encoded for 983 amino acids (SEQ ID NO: 7). Despite deletion of large portion of mucin-like domain, approximately ten KEPAPTT-like peptide repeats remained. Importantly, none of these are identical to the canonical human repeat sequence, but even if they had been, a skilled person could not have predicted whether delivery of the shortened canine Lub1 would be effective in treating OA. These repeats are thought to be important for lubrication properties as they are potential O-linked oligosaccharide attachment sites. The codon-optimization of this shorter lubricin (Lubico; SEQ ID NO: 6) increased the GC content from 44% to 60% and had 74% nucleotide similarity to the original canine DNA sequence. This shorter canine cDNA was then used to generate a plasmid expression cassette with minCBA promoter, cLubico and BGHpA (FIG. 10). Expression plasmids with 6×His-tags and modifications in putative ATG nucleotide sequences in the intron region (to minimize false translational start sites) were also made.

### Canine Lubricin Expression Analysis.

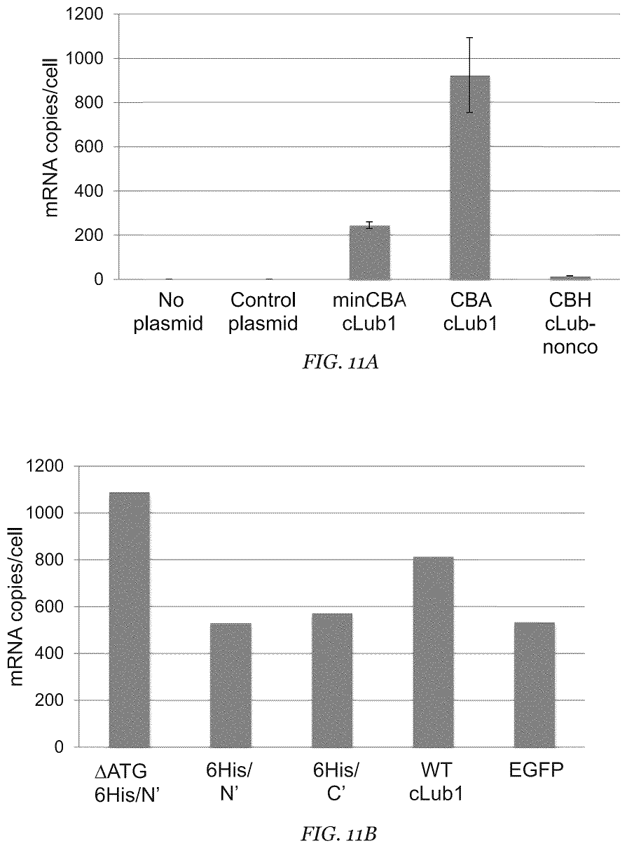

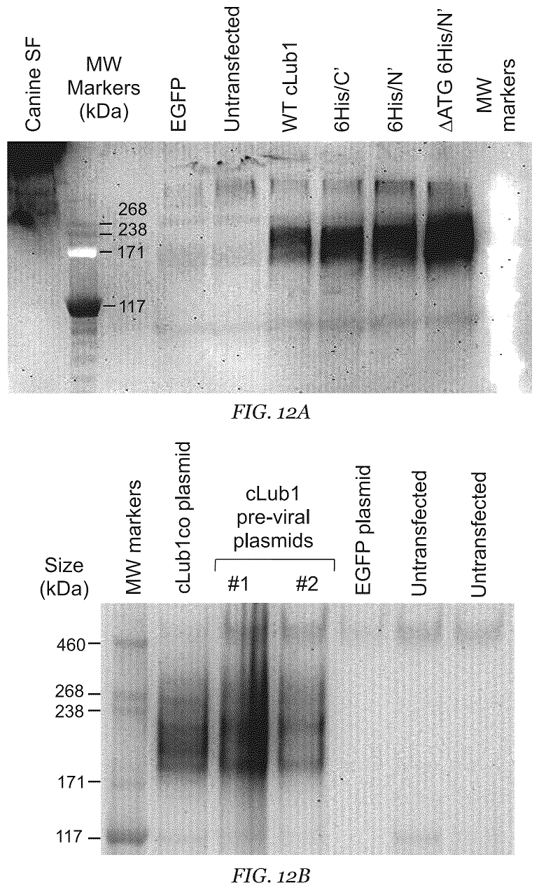

Expression of cLubico from the minimal CBA promoter (minCBA-cLubico) plasmid was confirmed in vitro by demonstrating increased mRNA levels in transfected 293 cells (FIG. 11A). The activity from the minCBA-cLubico construct with the shorter intron was about 3-fold lower than using the full-length CBA-HI construct (CBA-cLubico). Very little transcription was observed with plasmid containing the full-length lubricin and non-codon optimized construct (CBH-cLubr). Transcript analysis was also performed for expression cassettes with various modifications (FIGS. 10, 11B). Expression from the minCBA-Lubico was comparable to expression of EGFP and construct with C-terminal 6×His-tag. Deletion of the putative two ATG codons present in the hybrid intron appeared to enhance expression levels about 2-fold. Additional morphological changes observed in Lubico transfected cells also suggested Lub1 expression as these changes were not present in the un-transfected or EGFP-plasmid transfected cells (not shown).

Production of canine Lub1 protein from the various expression plasmids was tested by Western blot analysis using an antibody to lubricin and showed a protein of 250-380 kDa in the concentrated culture media (FIG. 12A). The expected size based on 1339 amino acid is approximately 160 kDa but the larger and diffuse pattern of the signal is likely due to glycosylation. Little detection was seen in un-transfected or EGFP plasmid transfected cells. Additionally, the ΔATG modification appeared to increase lubricin detection similar to observed for elevated transcript levels from this constructs. Protein expression was also confirmed from pre-viral AAV plasmid and showed comparable protein detection (FIG. 12B). In summary, these results demonstrate that the plasmids with canine lubricin expression cassettes expressed and secreted glycosylated lubricin protein.

Generation of rAAV Vector with Canine Lubricin Expression Cassette.

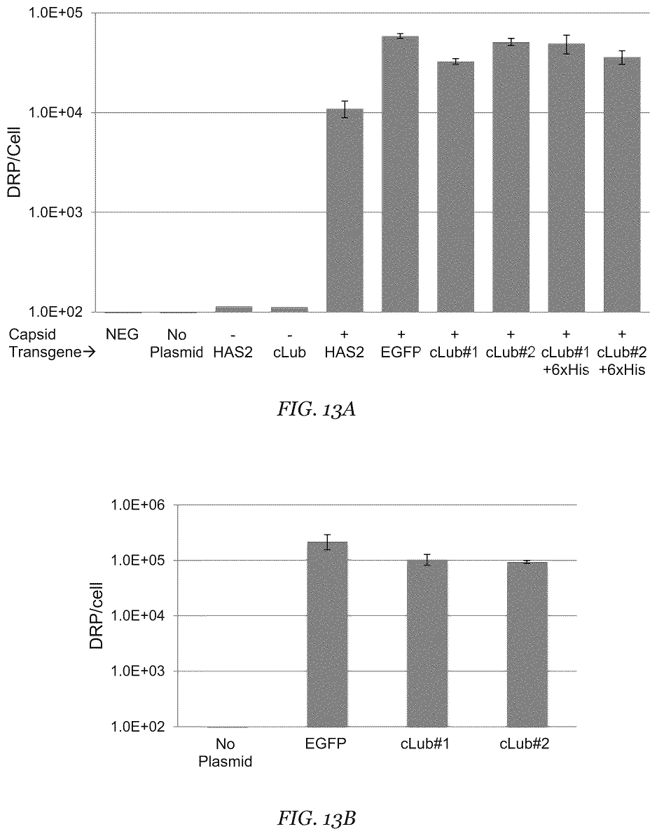

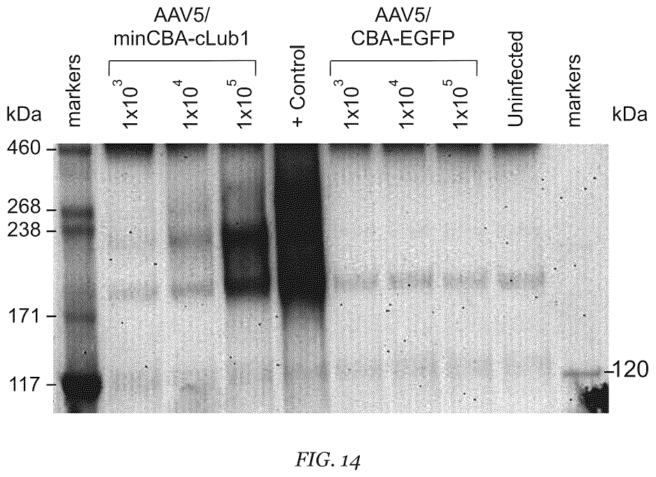

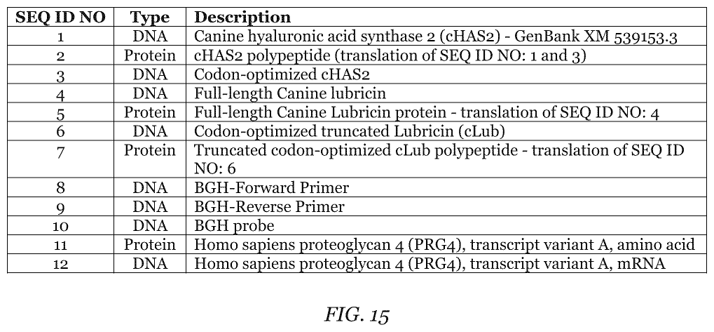

Having confirmed the canine lubricin expression from plasmid vectors, we next tested whether the expression cassette can be packaging into AAV2 and AAV5 capsid serotypes in a small-scale packaging experiment (FIGS. 13A, B). The data showed comparable packaging efficiency of canine lubricin both with AAV2 and AAV5 capsids as was observed for EGFP expression vectors. Inclusion of 6×His-tag did not alter rAAV vector yields. About 5-fold lower level of packaging was measured for AAV2 vector with a canine HAS2 expression cassette. A research-scale production of AAV5/minCBA-cLub1 was then performed to evaluate scaling-up vector production. The vector yield was comparable to that of standard AAV2 and AAV5 vectors with EGFP as transgene (data not shown). The rAAV5 vector was then tested for lubricin production and secretion in HEK293 cells in vitro. Analysis of conditioned media by Western blot demonstrated a dose-dependent detection of canine lubricin (FIG. 14). In summary, the data indicates that a shortened version of canine lubricin can be used to generate a rAAV vectors and that the cells infected with this vector can mediate lubricin synthesis and secretion into media.

### Example 2d—Conclusion

As indicated above, lubricin as a transgene presents a number of challenges for rAAV generation. First, the size of lubricin cDNA with necessary expression elements exceeds the rAAV packaging capacity and thus, a shorter cDNA version was required. Interestingly, compared to the human lubricin amino acid sequence in the mucin-like domain, no perfect KEPAPTT-repeats exists in the canine sequence (FIG. 16). For generation of a recombinant rAAV vector, any repeating DNA sequences could pose a challenge as repeat sequences can reduce the stability and integrity of virus genomes by causing DNA deletions and rearrangements during virus production. However, the disclosed (and surprising) results indicated that the generation of rAAV virus containing and expressing the novel canine lubricin sequence was feasible, considering that comparable vector yields were obtained when compared to standard EGFP reporter vectors. Furthermore, cells infected with the disclosed vector both produced and secreted canine lubricin. Accordingly, this a first report demonstrating a single rAAV vector strategy for lubricin gene delivery.

### Example 3—In Vivo Efficacy Study of AAV-HAS2 in Medial Meniscal Ligament Release (MMR) Model

The objective of this study was to evaluate HA synthase-2 gene therapy efficacy using gross observations and histology of the canine OA stifle model. Twelve purpose-bred intact male mongrel dogs (foxhound phenotype, ˜20-23 kg) were anesthetized and the medial meniscal ligament release (MMR) of the right stifle was accomplished arthroscopically (d-14).

Phosphate buffered saline (PBS control) or 5×10¹¹ DNase resistant particles [drp] of recombinant AAV5 carrying canine hyaluronic acid synthase 2 (cHAS-2), were administered intra-articularly on Day o (n=6 dogs/group).

Plasma was collected on Days 0 and 182 from all dogs for joint inflammation biomarker levels. Right and left synovial fluid was collected for HA level analysis on Days 0, 56, 112 and 182 from all PBS control and cHAS-2 treatment groups.

Dogs were euthanized on Day 182, and the cartilage defect induced by meniscal ligament release (indicated by India ink staining) was measured and joint tissues were collected for histopathology according to OA Research Society International (OARSI) standard techniques.

Gross and histologic data were analyzed using Kruskal-Wallis with GraphPad Prism 6 statistical software.

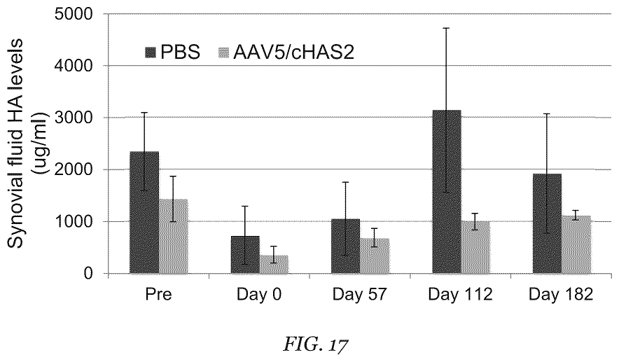

Total HA levels in synovial fluids were measured and did not show any treatment-related differences in total HA levels (FIG. 17).

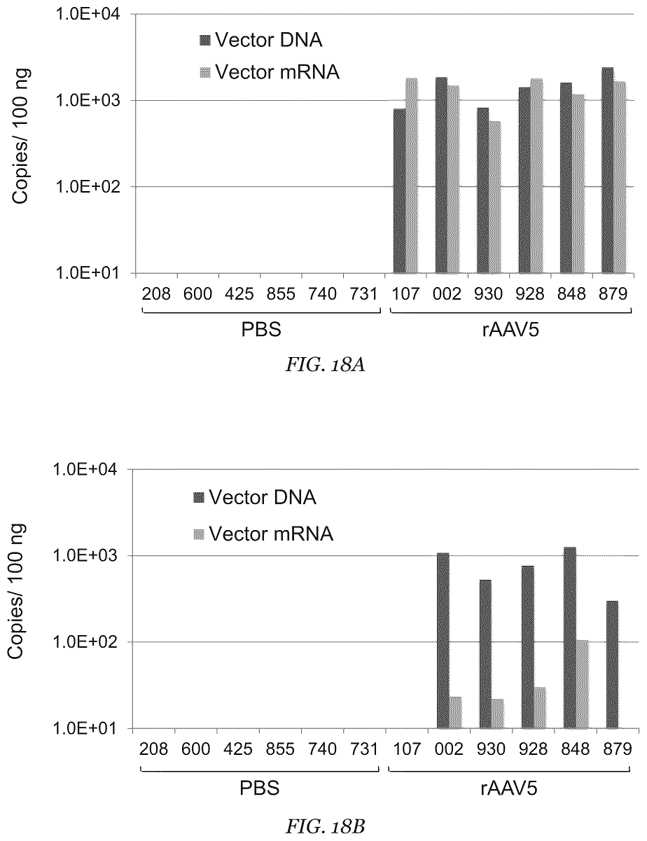

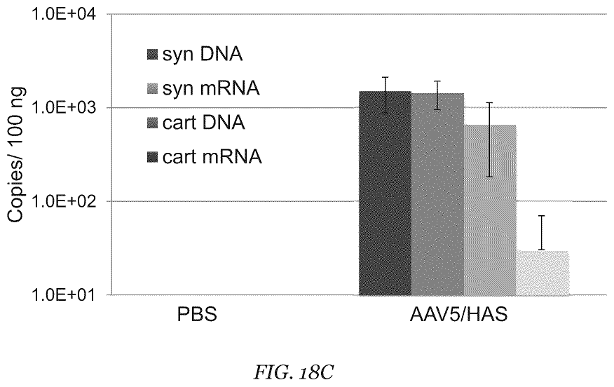

Synovial and cartilage samples were collected from the treated joints on day 182 and analyzed for detection of viral genomes (FIG. 18A). Vector derived DNA and mRNA were detected in each individual rAAV5/cHAS-2 injected joint in the synovial (FIG. 18A) and the majority of the cartilage (FIG. 18B) samples. The data is summarized in FIG. 18 C showing group average for vector genomes and mRNA in both tissue samples.

There was no evidence of local or systemic toxicity associated with intra-articular administration of HA synthase-2 gene therapy. There was a consistent preservation of the cartilage structure in the cHAS-2 treatment group compared to PBS-treatment. Reduced size and depth of lesions on both medial femoral condyle and medial tibial plateau joint surfaces were more pronounced in the femoral condyle in four of the six rAAV5/cHAS2-treated dogs.

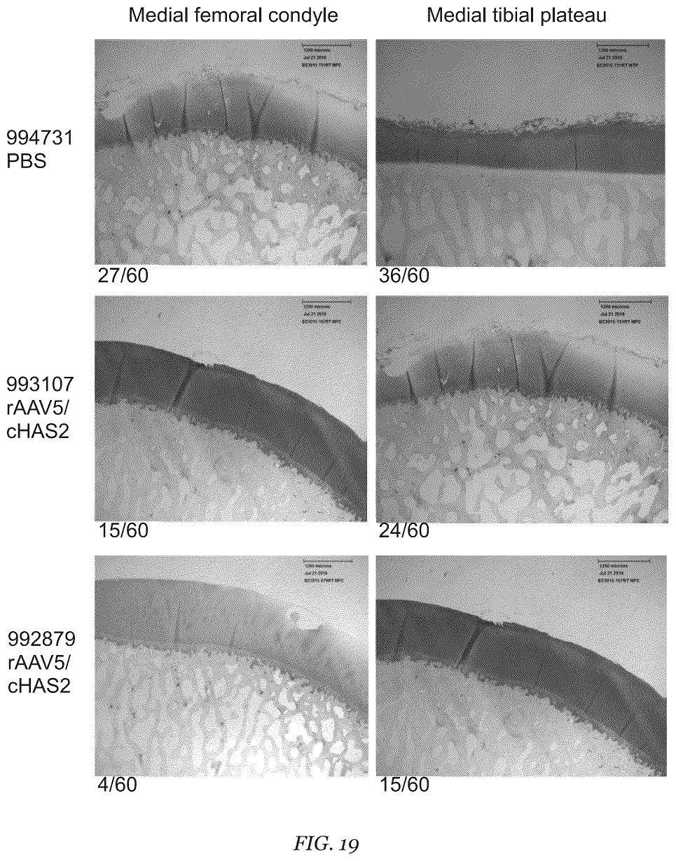

In FIG. 19, the histopathology score based on Cook et al. (2001) is shown in the lower left corner for each medial femoral condyle and medial tibial cartilage images (2×). Dog 994731/PBS had widespread erosion down to the middle zone with considerable loss of proteoglycan in both cartilage surfaces. No chondroprotective effect was observed. Dog 993107, treated with rAAV5/cHAS2, had shallow lesion in the superficial zone of the femoral cartilage, but overall, there was a good preservation of the rest of the cartilage and little loss of proteoglycan. The tibial plateau lesion was deeper into the middle zone with moderate proteoglycan depletion. A chondroprotective effect was observed in femoral condyle as the underlying cartilage is relatively normal. Dog 992879 treated with rAAV5/cHAS2 had some proteoglycan loss in femoral cartilage but the overall morphology was preserved. The tibial plateau had a well-defined focal erosion but the majority of the cartilage was preserved. Thus, there was some evidence of chondroprotection as the lesions were smaller and less severe.

Notably, one of the rAAV5-treated animals having no detectable vector in the cartilage sample also had the largest tibial plateau lesion area (dog 993107, FIG. 19). Conversely, one of the rAAV5-treated animals (dog 992879) having vector detected both in synovium and cartilage, but lacking mRNA detection the in cartilage, had the best cartilage structure.

Accordingly, the presence of the rAAV5-HAS2 vector is associated with the best cartilage structure, and, its absence is associated with the largest tibial plateau lesion area. Thus, despite variability in vector/mRNA detection, the rAAV5 vector expressing HAS2 appears to have elicited the desired clinical result.

Taken together, the results confirmed consistent rAAV5-mediated gene transfer into synovium and cartilage of canine OA joints and demonstrated sustained vector derived expression for at least six months. Histological analysis indicated reduced cartilage pathology and delayed disease progression in the majority of the cHAS-2 treated joints while little differences were observed in the total HA levels in the synovial fluid. The latter may indicate that local expression of HA in the cartilage and synovial tissues had some disease modifying properties without elevating total synovial fluid HA levels. Alternatively, changes in the molecular weight of HA synthesized that could not be detected by measuring total HA levels may have also contributed to beneficial effects by the rAAV5/cHAS-2.

### REFERENCES

Sanderson R O et al. Systematic review of the management of canine osteoarthritis. Veterinary Record (2009) 164, 418-424
McIlwraith C W. Frank Milne Lecture: from arthroscopy to gene therapy: 30 years of looking in joints. Am Assoc Equine Pract 2005; 51:65-113.
Cook et al. The OARSI histopathology initiative—recommendations for histological assessments of osteoarthritis in the dog. Osteoarthritis Cartilage, 2010; 18 suppl 3:S66-79.

The invention is further described in the following numbered paragraphs:
1. A method of treating a mammalian subject suffering from osteoarthritis (OA), comprising intra-articularly administering to said mammalian subject a therapeutically effective amount of a recombinant adeno-associated virus (rAAV) comprising a nucleic acid encoding an osteo-protective or osteo-regenerative polypeptide operably linked to a promoter, wherein the polypeptide is expressed in vivo in the mammalian subject in an amount effective to alleviate the symptoms of OA.
2. The method of paragraph 1, wherein the polypeptide is a hyaluronic acid synthase (HAS), i a lubricin, an Interleukin-1 Receptor (IL-1R) antagonist, an Insulin-like growth factor 1 (IGF-1), a fibroblast growth factor 2 (FGF-2), a Transforming growth factor beta 1 (TGFβ1), a Bone Morphogenetic protein 7 (BMP7), a Glucosamine-fructose-6-phosphate aminotransferase (GFAT), an Interleukin 10 (IL-10), a heme oxygenase-1 HO-1, biologically active truncations thereof, or combinations thereof.
3. The method of paragraph 1 or 2, wherein the polypeptide is a HAS2 polypeptide.
4. The method of any one of paragraphs 1-3, wherein the mammalian subject is a human, canine or feline.
5. The method of any one of paragraphs 1-4, wherein the mammalian subject is a canine. 6. The method of paragraph 5, wherein the polypeptide is canine HAS2.
7. The method of paragraph 5 or 6, wherein the HAS2 polypeptide comprises an amino acid sequence having at least 90% identity to the amino acid sequence as set forth in SEQ ID NO: 2, or a fragment, a variant, or a homolog thereof which exhibits HAS2 activity in vivo in the subject.
8. The method of any one of paragraphs 5-7, wherein the HAS2 polypeptide comprises the amino acid sequence set forth in SEQ ID NO: 2.
9. The method of any one of paragraphs 5-8, wherein the nucleic acid encoding the HAS2 polypeptide has a nucleotide sequence having at least 90% identity to the nucleotide sequence set forth in SEQ ID NO: 3.
10. The method of any one of paragraphs 5-9, wherein the rAAV comprises a rAAV vector genome comprising from 5′ to 3′ the following elements: 5′ AAV inverted terminal repeat (ITR), stuffer nucleic acid, a promoter, an intron (IN), a cHAS2 codon-optimized cDNA, a polyadenylation signal (pA), and a 3′ AAV ITR.
11. The method of paragraph 10, wherein the promoter is a chicken beta-actin (CBA) promoter.
12. The method of paragraph 1 or 2, wherein the polypeptide is a lubricin polypeptide.
13. The method of paragraph 12, wherein the lubricin polypeptide comprises an amino acid sequence having at least 90% identity to the amino acid sequence set forth in SEQ ID NO: 7, or a fragment, a variant, or a homolog thereof which exhibits Lubricin activity in vivo in the subject.
14. The method of paragraph 13, wherein the lubricin polypeptide comprises the amino acid sequence set forth in SEQ ID NO: 7.
15. The method of paragraph 13 or 14, wherein the nucleic acid encoding the lubricin polypeptide has a nucleotide sequence having at least 90% identity to the nucleotide sequence set forth in SEQ ID NO: 6.
16. The method of any one of paragraphs 13-14, wherein the rAAV comprises a rAAV vector genome encoded by plasmid pITR/minCBA-HI-cLubico-BGH.
17. The method of any of paragraphs 1-9 or 12-16, wherein the promoter is selected from the group consisting of a CMV IE promoter, a RSV promoter, an HSV-1 TK promoter, a SV40 early promoter, a SV40 late promoter, an adenovirus major late promoter, a phosphoglycerate kinase gene promoter, a metallothionein gene promoter, an α-1 antitrypsin gene promoter, an albumin gene promoter, a collagenase gene promoter, an elastase I gene promoter, a β-actin gene promoter, a CBA promoter, a β-globin gene promoter, a γ-globin gene promoter, an α-fetoprotein gene promoter, and a muscle creatine kinase (CK) gene promoter.
18. The method of paragraph 1, wherein the AAV comprises an AAV2 or an AAV5 capsid.
19. A method of increasing the production of hyaluronic acid in chondrocytes and/or synoviocytes of a canine, comprising the steps of administering a rAAV to the canine, wherein the rAAV comprises a rAAV vector genome comprising nucleic acid encoding an HAS2 enzyme operably linked to a promoter, and wherein following administration the HAS2 enzyme is expressed and catalyzes the production of additional hyaluronic acid, thereby increasing the level of hyaluronic acid (HA) in the canine.
20. The method of paragraph 19, wherein the HAS2 is produced in sufficient quantity to treat the symptoms of OA in canine.
21. The method of paragraph 20, wherein the HA levels are restored to levels found in healthy canines.
22. A method of treating a canine suffering from OA, comprising administering to the canine a therapeutically effective amount of rAAV, wherein the rAAV comprises an AAV vector genome comprising a nucleic acid encoding a HAS2 operably linked to a promoter.
23. A method of treating a human suffering from OA, comprising, administering to the human a therapeutically effective amount of rAAV, wherein the rAAV comprises an AAV vector comprising nucleic acid encoding a HAS2 operably linked to a promoter.
24. The method of any one of paragraphs 19-23, wherein the nucleic acid encoding the HAS2 has at least 90% identity to the nucleotide sequence set forth in SEQ ID NO:3 or encodes an HAS2 that has an amino acid sequence at least 90% identical to the amino acid sequence of SEQ ID NO: 2.
25. The method of any one of paragraphs 19-23, wherein the HAS2 has the amino acid sequence set forth in SEQ ID NO: 2.
26. A method of increasing the production of a lubricin in chondrocytes and/or synoviocytes of a canine, comprising the steps of administering a rAAV to the canine, wherein the rAAV comprises a rAAV vector comprising nucleic acid encoding a lubricin operably linked to a promoter, and wherein following administration the lubricin is expressed thereby increasing the level of lubricin in the canine.
27. The method of paragraph 26, wherein the lubricin is produced in sufficient quantity to treat the symptoms of OA in canine.
28. The method of paragraph 26, wherein the lubricin levels are restored to levels found in healthy canines.
29. A method of treating a canine suffering from OA, comprising, administering to said canine a therapeutically effective amount of rAAV, wherein the rAAV comprises an rAAV vector genome comprising nucleic acid encoding a lubricin operably linked to a promoter.
30. A method of treating a human suffering from OA, comprising administering to said human a therapeutically effective amount rAAV, wherein the rAAV comprises an AAV vector genome comprising nucleic acid encoding a lubricin operably linked to a promoter.
31. The method of any one of paragraphs 26-30, wherein the nucleic acid encoding the lubricin polypeptide has at least 90% identity to the sequence set forth in SEQ ID NO: 6 or the nucleic acid encodes a lubricin that has an amino acid sequence at least 90% identical to the amino acid sequence set forth in SEQ ID NO: 7.
32. The method of any one of paragraphs 26-30, wherein the lubricin has the amino acid sequence set forth in SEQ ID NO: 7.
33. The method of any one of paragraphs 19-32, wherein the promoter is selected from the group consisting of a CMV IE promoter, a RSV promoter, an HSV-1 TK promoter, a SV40 early promoter, a SV40 late promoter, a phosphoglycerate kinase gene promoter, a metallothionein gene promoter, an α-1 antitrypsin gene promoter, an albumin gene promoter, a collagenase gene promoter, an elastase I gene promoter, a CBA promoter, a β-actin gene promoter, a β-globin gene promoter, a γ-globin gene promoter, an α-fetoprotein gene promoter, and a muscle creatine kinase gene promoter.
34. The method of any one of paragraphs 19-25, wherein the rAAV comprises a rAAV vector genome encoded by plasmid Ps-AAV-ITR/CBA-cHAS2CO-BGH.
35. The method of any one of paragraphs 26-32, wherein the rAAV comprises a rAAV vector genome encoded by plasmid Ps-AAV-ITR/minCBA-HI-cLubico-BGH.
36. A method of preventing the development of OA in a mammalian subject at risk thereof, comprising administering to said canine a therapeutically effective amount of rAAV, wherein the rAAV comprises an rAAV vector genome comprising nucleic acid encoding a HAS2 operably linked to a promoter.
37. The method of paragraph 36, wherein the nucleic acid encoding the HAS2 polypeptide has at least 90% identity to the sequence set forth in SEQ ID NO: 2 or encodes a HAS2 that has an amino acid sequence at least 90% identical to the amino acid sequence of SEQ ID NO: 2.
38. The method of paragraph 36 or 37, wherein the HAS2 polypeptide comprises the amino acid sequence set forth in SEQ ID NO: 3.
39. A method of preventing the development of OA in a mammalian subject at risk thereof, comprising administering to said canine a therapeutically effective amount of a rAAV, wherein the rAAV comprises a rAAV vector genome comprising nucleic acid encoding a lubricin operably linked to a promoter.
40. The method of paragraph 39, wherein the nucleic acid encoding the lubricin has at least 90% identity to the sequence as set forth in SEQ ID NO: 6 or the nucleic acid encodes a lubricin that has an amino acid sequence at least 90% identical to the amino acid sequence set forth in SEQ ID NO: 7.
41. The method of paragraph 40, wherein the lubricin polypeptide has the amino acid sequence set forth in SEQ ID NO: 7.
42. The method of any one of paragraphs 36-38, wherein the promoter is selected from the group consisting of a CMV IE promoter, a RSV promoter, an HSV-1 TK promoter, a SV40 early promoter, a SV40 late promoter, a phosphoglycerate kinase gene promoter, a metallothionein gene promoter, an α-1 antitrypsin gene promoter, an albumin gene promoter, a collagenase gene promoter, an elastase I gene promoter, a CBA promoter, a β-actin gene promoter, a β-globin gene promoter, a γ-globin gene promoter, an α-fetoprotein gene promoter, and a muscle creatine kinase gene promoter.
43. The method of paragraphs 26, wherein the rAAV comprises a rAAV vector genome encoded by plasmid Ps-AAV-ITR/CBA-cHAS2co-BGH.
44. The method of paragraphs 26, wherein the rAAV comprises a rAAV vector genome encoded by plasmid Ps-AAV-ITR/minCBA-HI-cLubico-BGH.
45. The method of any one of paragraphs 19-44, wherein the rAAV is administered intra-articularly.
46. A recombinant plasmid vector comprising a nucleic acid sequence encoding a canine HAS2 polypeptide operably linked to a promoter.
47. The recombinant plasmid of paragraph 46, wherein the nucleic acid sequence encoding the HAS2 polypeptide has at least 90% identity to the sequence as set forth in SEQ ID NO:3 or the nucleic acid encodes a HAS2 polypeptide comprising an amino acid sequence at least 90% identical to the amino acid sequence set forth in SEQ ID NO: 2.
48. The recombinant plasmid of paragraph 46 or 47, wherein the HAS2 polypeptide comprises the amino acid sequence set forth in SEQ ID NO: 2.
49. The recombinant plasmid of any one of paragraphs 46-49, comprising pCBA-HI-cHAS2-BGHpA.
50. A recombinant plasmid vector comprising a nucleic acid sequence encoding a shortened canine lubricin operably linked to a promoter.
51. The recombinant plasmid of paragraph 50, wherein the nucleic acid sequence encoding the lubricin has at least 90% identity to the nucleotide sequence set forth in SEQ ID NO: 6 or the nucleic acid encodes a lubricin comprising an amino acid sequence at least 90% identical to the amino acid sequence set forth in SEQ ID NO: 7.
52. The recombinant plasmid of paragraph 50 or 51, wherein the lubricin polypeptide has an amino acid sequence as set forth in SEQ ID NO: 7.
53. The recombinant plasmid of any one of paragraphs 46-49 or 50-52, wherein the promoter is selected from the group consisting of a CMV IE promoter, a RSV promoter, an HSV-1 TK promoter, a SV40 early promoter, a SV40 late promoter, a phosphoglycerate kinase gene promoter, a metallothionein gene promoter, an α-1 antitrypsin gene promoter, an albumin gene promoter, a collagenase gene promoter, an elastase I gene promoter, a CBA promoter, a β-actin gene promoter, a β-globin gene promoter, a γ-globin gene promoter, an α-fetoprotein gene promoter, and a muscle creatine kinase gene promoter.
54. A recombinant AAV5 viral vector comprising the nucleotide sequence set forth in SEQ ID NO: 8.
55. A rAAV comprising the rAAV vector of paragraph 53.
56. A pharmaceutical composition comprising the rAAV of paragraph 55, and at least one pharmaceutically or veterinarily acceptable carrier, excipient, or vehicle.
57. A method of treating a mammalian subject suffering from osteoarthritis, comprising, intra-articularly administering to said mammalian subject a therapeutically effective amount of the pharmaceutical composition of paragraph 56.
58. The method of paragraph 57, wherein the mammalian subject is a human or canine animal.
59. An adeno-associated virus (AAV)-based biological delivery and expression system for use in the treatment of OA in mammalian joints by long-term gene expression of HAS2 or lubricin in synovial and/or chondrocyte cells, comprising a rAAV, wherein the rAAV comprises a rAAV vector comprising a nucleic acid sequence encoding HAS2 or lubricin, left and right AAV inverted terminal repeats (L ITR and R ITR), and wherein the expression of the HAS2 or lubricin gene within synovial and/or chondrocyte cells is regulated by a promoter, which is located upstream of the reading frame of the nucleic acid sequence encoding for HAS2 or lubricin and which is specifically activated by increased levels of immune stimulatory substances.
60. The AAV system of paragraph 59, wherein the HAS2 is a mammalian HAS2.
61. The AAV system of paragraph 59 or 60, wherein the HAS2 is a human HAS2.
62. The AAV system of any one of paragraphs 59-61, wherein the promoter is an inflammation-inducible promoter.
63. The AAV system of paragraph 62, wherein the inducible promoter is selected from the following: an NF-KB promoter, an interleukin 6 (II-6) promoter, an interleukin-1 (11-1) promoter, a tumor necrosis factor (TNF) promoter, a cyclooxygenase 2 (COX-2) promoter, a complement factor 3 (C3) promoter, a serum amyloid A3 (SAA3) promoter, a macrophage inflammatory protein-1a (MIP-1a) promoter and hybrid constructs thereof.
64. The AAV system according to any one of paragraphs 59-63, wherein the rAAV vector genome comprises nucleic acid encoding a HAS2 comprising the amino acid sequence of SEQ ID NO: 2, a lubricin comprising the amino acid sequence of SEQ ID NO: 7, or a functional variant thereof.
65. The AAV system of any one of paragraphs 59-64, wherein the rAAV vector genome comprises a marker gene that allows monitoring of the vector genome in the synovial and/or chondrocyte cells.
66. The AAV system of any one of paragraphs 59-65, wherein the rAAV vector genome comprises a nucleic acid having at least 80% or 90% sequence identity to the nucleic acid sequence set forth in SEQ ID NO:3 or SEQ ID NO: 6.
67. The AAV system of any one of paragraphs 59-66, wherein the rAAV vector genome comprises a nucleic acid sequence set forth in SEQ ID NO:3 or SEQ ID NO: 6.
68. The AAV system of any one of paragraphs 59-67 for the treatment or prevention of osteoarthritis (OA).
69. A pharmaceutical composition, comprising the AAV system of any one of paragraphs 59-68.
70. A rAAV comprising a rAAV vector, wherein the rAAV vector comprises a nucleic acid sequence encoding a canine HAS2 polypeptide operably linked to a promoter.
71. The rAAV of paragraph 70, wherein the nucleic acid sequence encoding the HAS2 polypeptide has at least 90% identity to the sequence as set forth in SEQ ID NO:3 or the nucleic acid encodes a HAS2 polypeptide comprising an amino acid sequence at least 90% identical to the amino acid sequence set forth in SEQ ID NO: 2.
72. The rAAV of paragraph 70 or 71, wherein the HAS2 polypeptide comprises the amino acid sequence set forth in SEQ ID NO: 2.
73. A rAAV comprising a rAAV vector, wherein the rAAV vector comprises a nucleic acid sequence encoding a shortened canine lubricin operably linked to a promoter.
74. The rAAV of paragraph 73, wherein the nucleic acid sequence encoding the lubricin has at least 90% identity to the nucleotide sequence set forth in SEQ ID NO: 6 or the nucleic acid encodes a lubricin comprising an amino acid sequence at least 90% identical to the amino acid sequence set forth in SEQ ID NO: 7.
75. The rAAV of paragraph 73 or 74, wherein the lubricin polypeptide has an amino acid sequence as set forth in SEQ ID NO: 7.
76. The rAAV of any one of paragraphs 50-53, wherein the rAAV vector comprises the nucleotide sequence as set forth in SEQ ID NO: 8.
77. The rAAV of any one of paragraphs 71-72 or 74-76, wherein the promoter is selected from the group consisting of a CMV IE promoter, a RSV promoter, an HSV-1 TK promoter, a SV40 early promoter, a SV40 late promoter, a phosphoglycerate kinase gene promoter, a metallothionein gene promoter, an α-1 antitrypsin gene promoter, an albumin gene promoter, a collagenase gene promoter, an elastase I gene promoter, a CBA promoter, a β-actin gene promoter, a β-globin gene promoter, a γ-globin gene promoter, an α-fetoprotein gene promoter, and a muscle creatine kinase gene promoter.
78. The rAAV of any one of paragraphs 71-77, wherein the rAAV comprises an AAV2 capsid or a AAV5 capsid.
79. A pharmaceutical composition comprising the rAAV of any one of paragraphs 71-78, and at least one pharmaceutically or veterinarily acceptable carrier, excipient, or vehicle.
80. A method of treating a mammalian subject suffering from osteoarthritis, comprising, intra-articularly administering to said mammalian subject a therapeutically effective amount of the pharmaceutical composition of paragraph 79.
81. The method of paragraph 80, wherein the mammalian subject is a human or canine animal.
82. An isolated nucleic acid having the sequence set forth in SEQ ID NO: 4.
83. An isolated polypeptide having the sequence set forth in SEQ ID NO: 5.

The invention will now be detailed according to the following set of non-limiting claims.

1-2. (canceled)

3: A recombinant adeno-associated virus (rAAV) comprising a rAAV vector, wherein the rAAV vector comprises a nucleic acid sequence encoding a canine lubricin operably linked to a promoter.

4: The rAAV of claim 3, wherein the nucleic acid sequence encoding the canine lubricin polypeptide has at least 90% identity to the nucleotide sequence set forth in SEQ ID NO:6 or the nucleic acid sequence encodes a canine lubricin polypeptide comprising an amino acid sequence at least 90% identical to the amino acid sequence set forth in SEQ ID NO: 7.

5: The rAAV of claim 4, wherein the canine lubricin polypeptide has an amino acid sequence as set forth in SEQ ID NO: 7.

6: The rAAV of claim 4, wherein the rAAV vector comprises the nucleotide sequence as set forth in SEQ ID NO: 8.

7: The rAAV of claim 3, wherein the promoter is selected from the group consisting of a CMV IE promoter, a RSV promoter, an HSV-1 TK promoter, a SV40 early promoter, a SV40 late promoter, a phosphoglycerate kinase gene promoter, a metallothionein gene promoter, an α-1 antitrypsin gene promoter, an albumin gene promoter, a collagenase gene promoter, an elastase I gene promoter, a CBA promoter, β-actin gene promoter, β-globin gene promoter, a γ-globin gene promoter, an α-fetoprotein gene promoter, and a muscle creatine kinase gene promoter.

8: The rAAV of claim 3, wherein the rAAV comprises an AAV2 capsid or a AAV5 capsid.

9: The rAAV of claim 8, wherein the rAAV comprises an AAV5 capsid.

10: A pharmaceutical composition comprising the rAAV of claim 3, and at least one pharmaceutically or veterinarily acceptable carrier, excipient, or vehicle.

11: A method of treating a mammalian subject suffering from osteoarthritis (OA), comprising intra-articularly administering to said mammalian subject a therapeutically effective amount of a recombinant adeno-associated virus (rAAV) comprising a nucleic acid encoding a canine lubricin polypeptide operably linked to a promoter, wherein the canine lubricin polypeptide is expressed in vivo in the mammalian subject in an amount effective to alleviate the symptoms of OA.

12-20. (canceled)

21: The method of claim 11, wherein the nucleic acid sequence encoding the canine lubricin polypeptide has at least 90% identity to the nucleotide sequence set forth in SEQ ID NO:6 or the nucleic acid sequence encodes a canine lubricin polypeptide comprising an amino acid sequence at least 90% identical to the amino acid sequence set forth in SEQ ID NO: 7.

22: The method of claim 21, wherein the canine lubricin polypeptide has an amino acid sequence as set forth in SEQ ID NO: 7.

23: The method of claim 21, wherein the rAAV vector comprises the nucleotide sequence as set forth in SEQ ID NO: 8.

24: The method of claim 11, wherein the promoter is selected from the group consisting of a CMV IE promoter, a RSV promoter, an HSV-1 TK promoter, a SV40 early promoter, a SV40 late promoter, a phosphoglycerate kinase gene promoter, a metallothionein gene promoter, an α-1 antitrypsin gene promoter, an albumin gene promoter, a collagenase gene promoter, an elastase I gene promoter, a CBA promoter, β-actin gene promoter, β-globin gene promoter, a γ-globin gene promoter, an α-fetoprotein gene promoter, and a muscle creatine kinase gene promoter.

25: The method of claim 11, wherein the rAAV comprises an AAV2 capsid or a AAV5 capsid.

26: A method of preventing the development of osteoarthritis (OA) in a mammalian subject at risk thereof, comprising administering to said subject a therapeutically effective amount of a recombinant adeno-associated virus (rAAV) comprising an rAAV vector, wherein the rAAV vector comprises a nucleic acid encoding a canine lubricin polypeptide operably linked to a promoter.

27: The method of claim 26, wherein the nucleic acid sequence encoding the canine lubricin polypeptide has at least 90% identity to the nucleotide sequence set forth in SEQ ID NO:6 or the nucleic acid sequence encodes a canine lubricin polypeptide comprising an amino acid sequence at least 90% identical to the amino acid sequence set forth in SEQ ID NO: 7.

28: The method of claim 27, wherein the canine lubricin polypeptide has an amino acid sequence as set forth in SEQ ID NO: 7.

29: The method of claim 27, wherein the rAAV vector comprises the nucleotide sequence as set forth in SEQ ID NO: 8.

30: The method of claim 26, wherein the promoter is selected from the group consisting of a CMV IE promoter, a RSV promoter, an HSV-1 TK promoter, a SV40 early promoter, a SV40 late promoter, a phosphoglycerate kinase gene promoter, a metallothionein gene promoter, an α-1 antitrypsin gene promoter, an albumin gene promoter, a collagenase gene promoter, an elastase I gene promoter, a CBA promoter, β-actin gene promoter, β-globin gene promoter, a γ-globin gene promoter, an α-fetoprotein gene promoter, and a muscle creatine kinase gene promoter.

31: The method of claim 26, wherein the rAAV comprises an AAV2 capsid or a AAV5 capsid.
